(19) United States
(12) Patent Application Publication
(10) Pub. No.: US 2024/0117048 A1
(43) Pub. Date: Apr. 11, 2024

(54) POLYPEPTIDE

(71) Applicant: UCL Business Ltd, London (GB)

(72) Inventors: Paul Maciocia, London (GB); Martin Pulé, London (GB)

(21) Appl. No.: 18/267,835

(22) PCT Filed: Dec. 15, 2021
(86) PCT No.: PCT/GB2021/053315

§ 371 Date: Jun. 16, 2023

(30) Foreign Application Priority Data
Dec. 16, 2020 (GB) 2019879.2

(51) Int. Cl. C07K 16/28; C07K 14/005

(52) U.S. Cl. CPC C07K 16/2809; C07K 14/005; C07K 2317/569; C07K 2319/02

(57) ABSTRACT

The present invention provides a polypeptide comprising: (i) a first domain which is capable of downregulating cell surface expression of an MHC class I molecule; and (ii) a second domain which is capable of binding to a target molecule.

### FIELD OF THE INVENTION

The present invention relates to a polypeptide for simultaneous downregulation of cell surface expression of both native MHC class I and a native target molecule (e.g. a native TCR) in an engineered cell. The polypeptide comprises a first domain which is capable of downregulating cell surface expression of an MHC class I molecule and a second domain which is capable of binding a component of TCR/CD3 complex or MHC class II. Also provided are an engineered cell comprising the polypeptide and therapeutic uses thereof as well as a method for making an engineered cell.

### BACKGROUND TO THE INVENTION

Adoptive immunotherapy with engineered cells (e.g. T-cells or NK cells) is a promising new clinical strategy. T-cells and NK cells engineered with e.g. chimeric antigen receptors (CARs) or transgenic T-cell receptors (TCRs) are promising cancer treatments. Chimeric antigen receptor (CAR) cells graft the specificity of a monoclonal antibody (mAb) to a cell, e.g. a T-cell.

Currently, most of these adoptive immunotherapy treatments are autologous, i.e. manufactured individually for each patient from the patient's own lymphocytes. This is effective and simple but has a number of disadvantages including (1) the need for apheresis from each patient, (2) delay in treating the patient may mean that there is insufficient time due to rapid progression of the patient's illness to generate an autologous product, (3) manufacturing failure in approximately 10% of cases due to insufficient quantity or quality of lymphocytes consequent to disease or chemotherapy, and (4) lack of economies of scale due to the requirement for bespoke product to be manufactured for each patient.

Generating engineered cells (e.g. T-cells or NK cells) independently of the patient (“off-the-shelf” engineered cells) is an alternative approach in which engineered cell products are manufactured from healthy donor lymphocytes. Depending on the process, economies of scale can be brought to bear on manufacture. Importantly, given the wide variability of human leukocyte antigen (HLA) types, for such a strategy to be practicable, engineered cell products will not be HLA-matched with the recipient, and in some cases will be completely HLA-mismatched from the recipient.

The generation of allogeneic engineered cells from a non-H LA-matched healthy donor brings its own technical challenges. One major challenge is that of graft-versus-host disease (GvHD), in which the native T-cell receptor (TCR) of the allogeneic engineered T-cell product recognises antigens in recipient tissues and starts attacking normal tissue. GvHD typically occurs in the setting of allogeneic haematopoietic stem cell transplantation (HSCT) where the donor and recipient are fully or partially matched. A more severe type of GvHD occurs when donor and recipient are not matched. This is known as “transfusion-associated GvHD” or ta-GvHD. A second major challenge to this approach is the potential for rejection of the allogeneic engineered cells by the recipient's immune system, termed host-versus-graft rejection (HvGR). In this situation, the engineered cells are rejected by the recipient's T-cells via recognition of allogeneic HLA on the engineered cells by the recipient's T-cells, thereby potentially limiting clinical efficacy of the adoptive immunotherapy.

One strategy to prevent or reduce both GvHD and HvGR is to block expression or function of both the native TCR and HLA molecules on the engineered cell. Knock-out of native TCR or HLA molecules can be achieved by using genome editing tools such as zinc-fingers, TALENs or CRISPR/Cas9 nucleases (Poirot, L. et al. (2015) Cancer Res. 75: 3853-3864; Provasi, E. et al. (2012) Nat. Med. 18: 807-815; Eyquem, J. et al. Nature (2017) 543: 113-117). In these approaches, the TCR or beta-2 globulin (a component of HLA) genes are disrupted, leading to permanent abrogation of TCR or HLA. However, this strategy has a number of challenges: (1) genome-editing methodologies have off-target effects; (2) double-stranded DNA breaks can lead to translocations when genome editing is performed at two loci; (3) the manufacturing process is more complex since both editing and transduction is required, i.e. a second gene-delivery is required in addition to the lentiviral/retroviral vector which provides the coding sequence for the CAR or TCR; (4) introduction of new genes and disruption of existing genes is uncoupled meaning the product can contained transduced, non-edited engineered cells or non-transduced, edited engineered cells, thereby reducing the yield; and (5) the process requires depletion of remaining TCR+ cells which is inefficient.

There is a need in the art for a method for the production of allogeneic engineered cells (e.g. T-cells or NK cells) for use in adoptive immunotherapy which avoids the aforementioned issues encountered with genome-editing methodologies.

### SUMMARY OF THE INVENTION

The present invention is predicated upon the development of a convenient strategy for simultaneous downregulation of cell surface expression of both native MHC class I and a native target molecule (e.g. a native TCR) in an engineered cell. This is achieved by expression of a single engineered fusion protein comprising a first domain which is capable of downregulating cell surface expression of an MHC class I molecule and a second domain which is capable of binding to a target molecule (e.g. to a component of the TCR/CD3 complex, thus preventing assembly of a complete TCR/CD3 complex). The first domain is capable of downregulating cell surface expression of an MHC class I molecule, for example by retention or re-targeting of the MHC class I molecule. When this first domain is fused with the second domain which is capable of binding to a target molecule, expression of the fusion protein achieves downregulated cell surface expression of both the native MHC class I molecule and the native target molecule. Advantageously, this fusion protein can be co-expressed, for example via a 2A peptide, with a CAR, transgenic TCR or suicide genes. Hence, any cell which is transduced and expresses a CAR, transgenic TCR or suicide genes also lacks cell surface expression of native MHC class I and native TCR molecules.

Thus, the present invention provides a convenient approach for the production of an allogeneic engineered cell in which a single molecule is co-expressed with e.g. a CAR to allow generation of an allogeneic CAR cell product. The approach employed in the invention is not associated with the disadvantages currently experienced with the existing, genome editing strategies to disruption of the native TCR. Advantages of this approach compared with genome-editing methodologies may be as follows: (1) reduced off-target effects; (2) no double-stranded breaks are generated and therefore there are no translocations; (3) the manufacturing process is more simple since introduction of the nucleic acid sequence encoding the CAR or transgenic TCR and of the polynucleotide encoding the fusion protein for simultaneous downregulation of cell surface expression of native MHC class I and native TCR can be performed in a single gene-delivery (e.g. transduction); (4) introduction of new genes and disruption of existing genes is coupled, thereby maximising the yield; (5) incorporation of a marker gene (e.g. Q8) is possible which allows positive selection which is more stringent than depletion.

Hence, the present invention relates the provision of a “universal” engineered cell (for example a therapeutic T-cell, such as a CAR T-cell) for inclusion in an off-the-shelf immunotherapy product.

Accordingly, in one aspect, the invention provides a polypeptide comprising:
(i) a first domain which is capable of downregulating cell surface expression of an MHC class I molecule; and
(ii) a second domain which is capable of binding a target molecule.

In some embodiments, the target molecule is a component of T-cell receptor (TCR)/CD3 complex or MHC class II.

In some embodiments, the second domain is an antibody or a fragment thereof.

In some embodiments, the second domain is selected from an scFv, a full-length antibody, a single chain antibody fragment, a F(ab) fragment, a F(ab′)2 fragment, a F(ab′) fragment, a single domain antibody (sdAb), a VHH/nanobody and a nanobody.

In some embodiments, the second domain is an scFv.

In some embodiments, the second domain comprises: (i) a sequence comprising the complementarity determining regions (CDRs) as shown in any one of SEQ ID NO: 19, SEQ ID NO: 21, SEQ ID NO: 23 or SEQ ID NO: 25; or (ii) a sequence as set forth in SEQ ID NO: 19, SEQ ID NO: 21, SEQ ID NO: 23, SEQ ID NO: 25 or SEQ ID NO: 42, or a sequence having at least 80% identity thereto or a functional fragment thereof.

In some embodiments, the second domain comprises: (i) a sequence comprising the CDRs as shown in SEQ ID NO: 19; or (ii) a sequence as set forth in SEQ ID NO: 19, or a sequence having at least 80% identity thereto or a functional fragment thereof.

In some embodiments, the polypeptide further comprises a signal peptide.

In some embodiments, the first domain is a viral protein which is capable of downregulating cell surface expression of an MHC class I molecule or a functional fragment thereof.

In some embodiments, the first domain is a viral protein, or a functional fragment thereof, which:
(i) induces MHC class I translocation from the ER to the cytosol and subsequent MHC class I degradation by the proteasome;
(ii) prevents transport of the MHC class I molecule from the ER to the plasma membrane; and/or
(iii) induces internalisation of the MHC class I molecule via endocytosis and subsequent degradation or sequestration of the MHC class I molecule.

In some embodiments, the first domain, when expressed in a cell, decreases cell surface expression of an MHC class I molecule by at least 10%.

In some embodiments, the first domain comprises human cytomegalovirus (HCMV) unique short 11 (US11), HCMV US2, adenovirus E3/19K (E19), HCMV US3, HCMV US10, human herpesvirus 7 (HHV-7) U21, human immunodeficiency virus type 1(HIV-1) negative factor (Nef), Kaposi's sarcoma-associated herpesvirus (KSHV) K3, KSHV K5, or a functional fragment thereof.

In some embodiments, the first domain comprises HCMV US11 or HCMV US2, or a functional fragment thereof.

In some embodiments, the first domain is HCMV US11 or a functional fragment thereof.

In some embodiments, the first domain comprises a sequence as set forth in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 43 or a sequence having at least 80% identity thereto or a functional fragment thereof.

In some embodiments, the first domain comprises a sequence as set forth in SEQ ID NO: 1 or SEQ ID NO: 3, or a sequence having at least 80% identity thereto or a functional fragment thereof.

In some embodiments, the first domain comprises a sequence as set forth in SEQ ID NO: 43 or a sequence having at least 80% identity thereto or a functional fragment thereof.

In some embodiments, the first domain is directly fused to the second domain. In other embodiments, the first domain is connected to the second domain by a linker sequence.

In a further aspect, the invention provides a polynucleotide encoding the polypeptide of the invention.

In a further aspect, the invention provides a nucleic acid construct comprising the polynucleotide of the invention.

In some embodiments, the nucleic acid construct further comprises a nucleic acid sequence encoding a chimeric antigen receptor (CAR) or a transgenic TCR, preferably wherein the nucleic acid construct further comprises a suicide gene.

In a further aspect, the invention provides a vector comprising the polynucleotide of the invention or the nucleic acid construct of the invention.

In a further aspect, the invention provides an engineered cell comprising the polypeptide of the invention, the polynucleotide of the invention, the nucleic acid construct of the invention or the vector of the invention.

In a further aspect, the invention provides a method for making an engineered cell according to the invention, which comprises the step of introducing:
(a) the polynucleotide of the invention, the nucleic acid construct of the invention or the vector of the invention and a nucleic acid sequence encoding a CAR or a transgenic TCR; or
(b) the nucleic acid construct of the invention or the vector of the invention;
   into a cell.

In some embodiments, the engineered cell is an allogeneic cell.

In some embodiments, the engineered cell is an engineered T cell or an engineered NK cell.

In some embodiments, the engineered cell further comprises a nucleic acid sequence encoding a CAR, a transgenic TCR and/or a suicide gene.

In some embodiments, the engineered cell has decreased cell surface expression of MHC class I and decreased expression of the target molecule compared to a cell which does not comprise the polypeptide of the invention, the polynucleotide of the invention, the nucleic acid construct of the invention or the vector of the invention.

In some embodiments, the cell is from a sample isolated from a donor.

In a further aspect, the invention provides a pharmaceutical composition comprising the polypeptide of the invention, the polynucleotide of the invention, the nucleic acid construct of the invention, the vector of the invention or an engineered cell of the invention.

In a further aspect, the invention provides a method for treating and/or preventing a disease comprising the step of administering an engineered cell of the invention, a population of engineered cells of the invention or the pharmaceutical composition of the invention to a recipient.

In a further aspect, the invention provides an engineered cell of the invention or a pharmaceutical composition of the invention for use in treating and/or preventing a disease.

In a further aspect, the invention provides the use of an engineered cell of the invention in the manufacture of a medicament for treating and/or preventing a disease.

In some embodiments, the donor is different to the recipient.

In some embodiments, the engineered cell is an allogeneic engineered cell or the population of engineered cells is a population of allogeneic engineered cells.

In some embodiments, the recipient is not HLA typed.

In some embodiments, the donor is not HLA matched to the recipient.

In some embodiments, the method comprises the following steps:
(i) isolation of a cell-containing sample from the donor;
(ii) transduction or transfection of the cells following the method according to claims 26; and
(iii) administering the cells obtained in (ii) to the recipient.

In some embodiments, the engineered cell is an allogeneic engineered cell or the population of engineered cells is a population of allogeneic engineered cells.

In some embodiments, the disease is a cancer.

In a further aspect, the invention provides a method of reducing or preventing graft versus host disease (GvHD) and host versus graft disease (HvGD) in a subject associated with the administration of one or more engineered cells to the subject, comprising the steps of:
(i) making the one or more engineered cells according to the method of the invention; and
(ii) administering the one or more engineered cells to the subject.

In a further aspect, the invention provides a method of reducing or preventing GvHD in a subject associated with the administration of one or more engineered cells to the subject, comprising the steps of:
(i) making the one or more engineered cells according to the method of the invention; and
(ii) administering the one or more engineered cells to the subject.

In a further aspect, the invention provides a method of reducing or preventing HvGD in a subject associated with the administration of one or more engineered cells to the subject, comprising the steps of:
(i) making the one or more engineered cells according to the method of the invention; and
(ii) administering the one or more engineered cells to the subject.

In some embodiments, the engineered cell is an allogeneic engineered cell.

In a further aspect, the invention provides a kit comprising the polynucleotide of the invention and a nucleic acid sequence encoding a CAR and/or a transgenic TCR, the nucleic acid construct of the invention or the vector of the invention.

In a further aspect, the invention provides a kit comprising:
(i) a vector comprising the polynucleotide of the invention and a vector comprising a nucleic acid sequence encoding a CAR or a transgenic TCR; or
(ii) a vector comprising the polynucleotide of the invention and a nucleic acid sequence encoding a CAR or a transgenic TCR.

### BRIEF DESCRIPTION OF THE FIGURES

FIG. 1: Downregulation of TCR and HLA-ABC with UCHT1-US11. T-cells from a healthy donor were mock transduced (NT, left) or transduced with a retroviral vector expressing UCHT1-US11 fusion protein with blue fluorescent protein (BFP) marker gene (right). Cells were stained for TCR (top) and HLA-ABC (bottom). Downregulation of both TCR and HLA-ABC was seen only in transduced cells. TCR=T-cell receptor, HLA=human leukocyte antigen, BFP=blue fluorescent protein. One representative donor shown, repeated in >10 donors.

FIG. 2: Single-step generation of TCR-negative, MHC-class 1-negative CAR T-cells. T-cells from a healthy donor were transduced with a construct combining RQR8 (CD34 and CD20-based sort-suicide gene), N-terminal FLAG-tagged anti-CD19 CAR and UCHT1-US11. (a) Schematic of expression vector containing anti-CD19 CAR, RQR8 sort-suicide gene and UCHT1-US11. (b) Co-expression of anti-CD19 CAR (staining for FLAG tag on CAR) with UCHT1-US11. (c) Purification of CAR-positive TCR-negative cells (a positive selection using CD34 magnetic beads) yielded >97% TCR-negative purity. (d) Down-regulation of HLA-ABC

FIG. 3: UCHT1-US11 fusion protein sequence.

FIG. 4: Downregulation of TCR and HLA-ABC with UCHT1-E19. Jurkat cells were transduced with a retroviral vector expressing UCHT1-E19 fusion protein with blue fluorescent protein (BFP) marker gene. Cells were stained for TCR (left) and HLA-ABC (right). Downregulation of both TCR and HLA-ABC was demonstrated. TCR=T-cell receptor, HLA=human leukocyte antigen, BFP=blue fluorescent protein.

FIG. 5: Downregulation of TCR and HLA-ABC with CD3VHH-US11. Primary human T cells were transduced with a retroviral vector expressing CD3VHH-US11 fusion protein with CD34 marker gene. Cells were stained for TCR (left) and HLA-ABC (right). Downregulation of both TCR and HLA-ABC was demonstrated. TCR=T-cell receptor, HLA=human leukocyte antigen.

### DETAILED DESCRIPTION OF THE INVENTION

### Polypeptide

Graft-versus-host disease (GvHD) occurs when the native TCR of the donor's T-cells recognise antigens in the recipient's healthy cells as foreign such that the donor's T-cells attack and damage the recipient's normal tissue. This can occur in the setting of haematopoietic stem cell transplantation (HSCT), bone marrow transplantation or adoptive immunotherapy with engineered cells, where the donor and recipient are fully or partially matched. A more severe type of GvHD occurs when donor and recipient are not matched.

Host-versus-graft rejection (HvGR) occurs when the donor's cells are rejected by the recipient's T-cells via recognition of allogeneic MHC on the donor's cells by the recipient's T-cells. This can also occur in the setting of haematopoietic stem cell transplantation (HSCT), bone marrow transplantation or adoptive immunotherapy with engineered cells and potentially limits clinical efficacy of the transplant or therapy. In HvGR, MHC class I and/or MHC class II molecules on the donor's cells can be recognised by the recipient's T cells.

Thus, adoptive immunotherapy with engineered cells (e.g. T-cells or NK cells) from a non-HLA-matched healthy donor (e.g. with allogeneic engineered T cells) has the potential for both GvHD and HvGR.

The present inventors have developed a strategy for simultaneous downregulation of cell surface expression of both native MHC class I and a native target molecule (e.g. a native TCR) in an engineered cell. This is achieved by expression of a single engineered fusion protein comprising a first domain which is capable of downregulating cell surface expression of an MHC class I molecule and a second domain which is capable of binding to a target molecule (e.g. to a component of the TCR/CD3 complex, thus preventing assembly of a complete TCR/CD3 complex). Thus, expression of the fusion protein (i.e. the polypeptide of the invention) may achieve downregulated cell surface expression of both the native HLA class I molecule and the native target molecule.

Thus, when the second domain is capable of binding MHC class II, cell surface expression of both MHC class I and MHC class II is simultaneously downregulated by the polypeptide of the invention. It will be appreciated by the skilled person that this strategy can prevent and/or reduce HvGR, for example in the setting of adoptive immunotherapy with allogeneic engineered cells.

Similarly, when the second domain is capable of binding a component of TCR/CD3 complex, cell surface expression of both MHC class I and TCR is simultaneously downregulated by the polypeptide of the invention. It will be appreciated by the skilled person that this strategy can prevent and/or reduce both HvGR and GvHD, for example in the setting of adoptive immunotherapy with allogeneic engineered cells.

Accordingly, the present invention provides a polypeptide comprising:
(i) a first domain which is capable of downregulating cell surface expression of an MHC class I molecule; and
(ii) a second domain which is capable of binding to a target molecule.

Preferably, the polypeptide is a fusion polypeptide. The first domain may be directly fused to the second domain. Alternatively, the first domain may be fused to the second domain by a linker sequence. Suitable linker sequences for fusing two domains are known in the art. Examples of suitable linker regions include, but are not limited to, peptide linkers or chemical linking groups.

Suitably, the linker may be a single amino acid, e.g. proline, which is suitable to separate the peptides.

Suitably, the first domain and second domain may be coupled by a flexible linker peptide.

Illustrative flexible linker peptides are glycine and/or serine-rich peptides. The peptide linker may comprise 4-20, 4-15, 4-10, 8-20 or 8-15 amino acids. Examples of suitable peptide linkers are known in the art and include, but are not limited to, GGSGGS (SEQ ID NO: 27), SGSGSGS (SEQ ID NO: 28), GGGGSGGGGS (SEQ ID NO: 29), GSGSGSGSGS (SEQ ID NO: 30), GGSGGSGGSGGS (SEQ ID NO: 31), and GGGGSGGGGSGGGGS (SEQ ID NO: 32).

Preferably, the linker sequence has the amino acid sequence SDP.

The linker may be a chemical linker comprising peptide coupling sites. As will be appreciated, the number of peptides coupled to the chemical linker may be defined—at least in part—by the number of peptide coupling sites provided by the chemical linker.

Accordingly, further suitable linkers include chemical linkers such as 8-Amino-3,6-dioxaoctanoyl or 18-Amino-4,7,10,13,16-pentaoxaoctadecanoyl.

Suitably, the first domain and second domain may be coupled to the chemical linker via covalent bonds at the peptide coupling sites.

Suitable chemical linkers with peptide coupling sites are known in the art.

The polypeptide may further comprise a signal peptide. The signal peptide may ensure that, when the polypeptide is expressed inside a cell, such as a T-cell, the nascent protein is directed to the ER. The signal peptide may be provided at the N-terminus of the polypeptide or second domain. Suitably, the signal peptide has the amino acid sequence METDTLLLWVLLVWIPGSTG (SEQ ID NO: 33).

Suitably, in embodiments of the invention wherein the luminal domain of the first domain is necessary or important for its function of downregulating cell surface expression of an MHC class I molecule (e.g. when the first domain comprises a US11, US2 or E19 polypeptide as described herein), the first domain is positioned at the C-terminus of the second domain so as to allow appropriate positioning of the luminal domain of the first domain. In other words, the fusion protein of the invention has the structure: N′—Second domain—First domain—C′.

### First Domain

The major histocompatibility complex (MHC) is a large genetic locus in the vertebrate genome containing a set of polymorphic genes that encode MHC class I and MHC class II molecules—cell surface proteins essential for the adaptive immune system. The MHC class I and MHC class II molecules bind a peptide derived from self-proteins (i.e. self antigens) or from pathogen (i.e. non-self proteins and antigens) and present the peptide on the cell surface for recognition by the appropriate T-cells. Human MHC class I and MHC class II are also called human leukocyte antigen (HLA).

The MHC class I antigen presentation pathway plays an important role in the recognition of pathogen infected cells, for example virally infected cells, by the immune system. MHC class I molecules are expressed on the cell surface of all nucleated cells and present on the cell surface peptide fragments derived from breakdown of intracellular proteins processed by the host cellular machinery. The presentation of self-antigens in association with MHC class I at the cell surface prevents an organism's immune system from targeting its own healthy cells. The presentation of non-self antigens in association with MHC class I at the cell surface results in recognition of the MHC-peptide complexes as foreign by circulating T-cells, and the infected cell is marked for destruction by the immune system. For example, in a virally infected cell, peptides derived from viral proteins may be presented by MHC class I at the cell surface. Cytotoxic T lymphocytes (CTL) monitor cell surface MHC class I molecules for peptides derived from viral proteins and eliminate infected cells. NK and NKT cells may also recognise cell surface MHC class I-peptide complexes.

MHC class I molecules are heterodimers of β₂-microglubulin and a heavy chain and undergo folding and assembly within the endoplasmic reticulum (ER) lumen. Peptide binding is the final stage of the assembly process. To facilitate peptide binding within the ER lumen, peptides are translocated from the cytosol into the ER lumen by the transporter associated with antigen processing (TAP), which also acts as a scaffold for peptide binding. After peptide binding, MHC class I molecules dissociate from TAP, transit to export sites on the ER membrane, are selectively incorporated into cargo vesicles for transport to the Golgi apparatus and migrate through the Golgi apparatus to the plasma membrane.

As used herein, the terms “MHC class I molecule” and “MHC class I” are used interchangeably to refer to heterodimers of β₂-microglubulin and a heavy chain which are encoded by the MHC locus. The HLAs corresponding to MHC class I are HLA-A, HLA-B and HLA-C.

In order to evade clearance by host immune cells, a number of viruses have evolved mechanisms to evade detection. In particular, several viruses encode proteins that interfere with the MHC class I pathway of antigen presentation, so viral peptides cannot be presented at the cell surface, via different mechanisms including inhibition of peptide loading to MHC class I via TAP and MHC class I re-targeting (including retention and sequestration) and degradation. Thus, many of these viral proteins downregulate cell surface expression of MHC class I molecules. Strategies for viral immune evasion are reviewed in Hewitt (2003) Immunology 110: 163-169.

One mechanism by which viral proteins interfere with the MHC class I pathway of antigen presentation is by the exploitation of the endogenous cellular quality control pathway that removes misfolded or unassembled proteins from the ER, termed the ER-associated protein degradation (ERAD) pathway. In the endogenous pathway, misfolded or unassembled proteins are exported from the ER into the cytosol where they undergo proteasomal degradation. However, binding of a viral protein has no obvious effect upon the conformation of the MHC class I heavy chain, but diverts the MHC class I heavy chain into the ERAD pathway. Thus, one mechanism by which viral proteins interfere with the MHC class I pathway of antigen presentation is by inducing MHC class I translocation from the ER to the cytosol and subsequent degradation by the proteasome.

Examples of viral proteins which interfere with the MHC class I pathway of antigen presentation in this manner include unique short 2 (US2) and US11 of human cytomegalovirus (HCMV). US2 and US11 are ER-localized type I integral membrane glycoproteins that promote rapid degradation of the MHC class I heavy chain by inducing its translocation from the ER to the cytosol and subsequent degradation by the proteasome. Translocation of the MHC class I heavy chain occurs soon after insertion and glycosylation of the MHC class I heavy chain in the ER.

MHC class I downregulation during HCMV infection is mediated largely by the transmembrane protein US11. US11 contains a luminal domain which binds MHC class I heavy chain in the ER, and a transmembrane domain which induces translocation of MHC class I heavy chain to the cytosol via TM EM129/Derlin1, where it undergoes proteasomal degradation (Lilley, B. N., et al. (2003) Mol. Biol. Cell 1: 3690-3698). Similarly, US2 contains a luminal domain which binds MHC class I heavy chain in the ER and US2 induces translocation of MHC class I heavy chain to the cytosol via Sec61, where it undergoes proteasomal degradation (Hewitt (2003) Immunology 110: 163-169). Thus, in cells infected by HCMV, expression of surface MHC class I molecules is downregulated, and HCMV-infected cells are not recognised as infected cells by circulating T-cells (Loureiro, J. & Ploegh, H. L. (2006) Advances in Immunology, 92: 225-305).

A further mechanism by which viral proteins interfere with the MHC class I pathway of antigen presentation is by directly binding to MHC class I and preventing transport of the MHC class I molecule to the plasma membrane, for example by retention in the ER or diversion to the lysosome. Examples of viral proteins which interfere with the MHC class I pathway of antigen presentation in this manner include adenovirus E3/19K (E19), HCMV US3 and US10, and human herpesvirus 7 (HHV-7) U21 (Hewitt (2003) Immunology 110: 163-169).

E19 is a type I integral membrane protein that directly binds MHC class I molecules in the ER and prevents their transport to the plasma membrane. E19 possesses two distinct mechanisms to inhibit MHC class I cell surface expression. Firstly, the cytosolic tail of E19 comprises a dilysine motif which is also present in the cytosolic tails of many ER-localized membrane proteins. Proteins carrying this motif are recognized by the coatamer protein complex (COP1) which is involved in retrograde transport of proteins from the Golgi apparatus to the ER. Thus, E19 binding of MHC class I causes MHC class I retention in the ER via a dilysine motif in the E19 cytosolic tail and thereby inhibits MHC class I trafficking to the cell surface. Secondly, E19 also binds to TAP and prevents TAP/MHC class I association.

HCMV US3 is an ER-localized type I integral membrane protein that transiently interacts with MHC class I molecules and inhibits the export of peptide-loaded MHC class I molecules from the ER. HCMV US10 is an ER-localized integral membrane glycoprotein, predicted to be a type I integral membrane protein, that impedes but does not block the export of MHC class I molecules from the ER.

HHV-7 U21 is a type I integral membrane glycoprotein that binds to properly folded MHC class I molecules in the ER and diverts them to the lysosome, thereby downregulating cell surface expression.

A further mechanism by which viral proteins interfere with the MHC class I pathway of antigen presentation is by inducing internalisation of the MHC class I molecule via endocytosis and subsequent degradation or sequestration. Examples of viral proteins which interfere with the MHC class I pathway of antigen presentation in this manner include human immunodeficiency virus type 1 (HIV-1) negative factor (Nef) and Kaposi's sarcoma-associated herpesvirus (KSHV) K3 and K5.

HIV-1 Nef is a myristoylated protein that mediates ADP-ribosylation factor 6 (ARF6)-dependent internalization of MHC class I molecules followed by sequestration in the trans-Golgi network. In the absence of Nef, the small GTPase ARF6 regulates a pathway of constitutive internalisation and recycling back to the plasma membrane of MHC class I molecules. Nef has been shown to accelerate the ARF6-dependent internalization of MHC class I molecules. Three motifs in the Nef sequence, which are not functionally equivalent, have been shown to be important for the downregulation of MHC class I cell surface expression: ₆₂EEEE₆₅, ₇₂PXXP₇₅ (where X can be any amino acid) and Met₂₀ within an amphiphatic α-helix. ₆₂EEEE₆₅ and ₇₂PXXP₇₅ are required for the internalisation of MHC Class I whereas Met₂₀ is required for the sequestration of MHC class I in the TGN. Thus, HIV-1 Nef directs MHC class I to the endosomal pathway, thereby downregulating cell surface expression.

KSHV K3 and K5 each cause rapid downregulation of cell surface expression of MHC class I molecules via clathrin-dependent endocytosis. K3 and K5 are integral membrane proteins which are E3 ubiquitin ligases that promote ubiquitination of MHC class I molecules along the secretory pathway. Ubiquitination by K3 and K5 targets MHC class I molecules for internalisation from the cell surface, translocation into the late endosomal pathway (i.e. into an acidic endocytic compartment) and degradation by acidic proteases. The cytosolic tail of the MHC class I heavy chain is reported to be the site of K3 and K5-induced ubiquitination. Thus, KSHV K3 and K5 directs MHC class I to the late endosomal pathway for degradation, thereby downregulating cell surface expression.

A further mechanism by which viral proteins interfere with the MHC class I pathway of antigen presentation is by inhibiting peptide loading onto MHC class I via TAP. The skilled person would understand that such viral proteins may not downregulate cell surface expression of MHC class I molecules. Thus, in one embodiment, the first domain is not a viral protein which interferes with the MHC class I pathway of antigen presentation solely by inhibiting peptide loading onto MHC class I via TAP.

An illustrative HCMV US11 amino acid sequence (Uniprot ID P09727-10) is shown as SEQ ID NO: 1.

SEQ ID NO: 1
MNLVMLILALWAPVAGSMPELSLTLFDEPPPLVETEPLPPLSDVSEYRVE
YSEARCVLRSGGRLEALWTLRGNLSVPTPTPRVYYQTLEGYADRVPTPVE
DVSESLVAKRYWLRDYRVPQRTKLVLFYFSPCHQCQTYYVECEPRCLVPW
VPLWSSLEDIERLLFEDRRLMAYYALTIKSAQYTLMMVAVIQVFWGLYVK
GWLHRHFPWMESDQW

Amino acids 1-17 of SEQ ID NO: 1 provide a signal peptide. Suitably the US11 amino acid sequence for use in the present invention may lack amino acids 1-17 of SEQ ID NO: 1.

An illustrative HCMV US11 nucleic acid sequence (European Nucleotide Archive CAA35278.1) is shown as SEQ ID NO: 2.

SEQ ID NO: 2
ATGAACCTTGTAATGCTTATTCTAGCCCTCTGGGCCCCGGTCGCGGGTAG
TATGCCTGAATTATCCTTGACTCTTTTCGATGAACCTCCGCCCTTGGTGG
AGACGGAGCCGTTACCGCCTCTGTCCGATGTTTCGGAGTACCGAGTAGAG
TATTCCGAGGCGCGCTGCGTGCTCCGATCGGGCGGTCGACTGGAGGCTCT
GTGGACCCTGCGCGGGAACCTGTCCGTGCCCACGCCGACACCCCGGGTGT
ACTACCAGACGCTGGAGGGCTACGCGGATCGAGTGCCGACGCCGGTGGAG
GACGTCTCCGAAAGCCTCGTCGCAAAACGCTACTGGCTCCGGGACTATCG
TGTTCCCCAACGCACAAAACTCGTGTTGTTCTACTTTTCCCCCTGCCACC
AATGCCAAACTTATTATGTAGAGTGCGAACCCCGGTGCCTCGTGCCTTGG
GTTCCCCTGTGGAGCTCGTTAGAGGACATCGAACGACTATTGTTCGAAGA
TCGCCGTCTAATGGCGTACTACGCGCTCACGATTAAGTCGGCGCAGTATA
CGCTGATGATGGTGGCAGTGATTCAAGTGTTTTGGGGGCTGTATGTGAAA
GGTTGGCTGCACCGACATTTTCCCTGGATGTTTTCGGACCAGTGGTGA

An illustrative HCMV US2 amino acid sequence is shown as SEQ ID NO: 3.

SEQ ID NO: 3
MNNLWKAWVGLWTSMGPLIRLPDGITKAGEDALRPWKSTAKHPWFEIED
NRCYIDNGKLFARGSIVGNMSRFVFDPKADYGGVGENLYVHADDVEFVP
GESLKWNVRNLDVMPIFETLALRLVLQGDVIWLRCVPELRVDYTSSAYM
WNMQYGMVRKSYTHVAWTIVFYSINITLLVLFIVYVTVDCNLSMMWMRF
FVC

An illustrative HCMV US2 nucleic acid sequence (NCBI Reference Sequence: NC_006273.2:c199930-199331) is shown as SEQ ID NO: 4.

SEQ ID NO: 4
ATGAACAATCTCTGGAAAGCCTGGGTGGGTCTTTGGACCTCCATGGGTC
CCTTGATCCGCCTGCCCGATGGCATCACTAAAGCCGGGGAAGACGCGCT
CCGGCCCTGGAAGTCCACGGCCAAGCACCCCTGGTTTGAGATCGAGGAC
AACCGGTGCTACATTGACAACGGCAAGTTGTTTGCTCGGGGGAGCATCG
TGGGCAACATGAGTCGGTTCGTCTTCGATCCGAAGGCCGATTATGGCGG
CGTGGGAGAGAACCTGTACGTACACGCCGACGACGTGGAGTTCGTTCCC
GGGGAGTCGTTAAAGTGGAACGTGCGGAACTTAGATGTGATGCCGATCT
TCGAGACGCTAGCCCTGCGTCTGGTACTGCAAGGGGATGTGATCTGGCT
GCGTTGCGTCCCCGAACTGCGAGTGGATTACACGTCTAGCGCGTACATG
TGGAACATGCAGTACGGGATGGTGCGGAAGTCATACACGCATGTGGCCT
GGACAATAGTGTTTTACTCCATAAACATTACCCTGTTGGTATTGTTTAT
CGTGTATGTGACTGTGGACTGTAACTTGTCTATGATGTGGATGCGGTTT
TTCGTGTGCTAA

An illustrative adenovirus E19 amino acid sequence (NCBI Reference Sequence: AP_000184.1) is shown as SEQ ID NO: 5 or SEQ ID NO: 43.

SEQ ID NO: 5
MRYMILGLLALAAVCSAAKKVEFKEPACNVTFKSEANECTTLIKCTTEHE
KLIIRHKDKIGKYAVYAIWQPGDTNDYNVTVFQGENRKTFMYKFPFYEMC
DITMYMSKQYKLWPPQKCLENTGTFCSTALLITALALVCTLLYLKYKSRR
SFIDEKKMP
SEQ ID NO: 43
TQKVDFKEPACNVTFAAEANECTTLIKCTTEHEKLLIRHKNKIGKYAVYA
IWQPGDTTEYNVTVFQGKSHKTFMYTFPFYEMCDITMYMSKQYKLWPPQN
CVENTGTFCCTAMLITVLALVCTLLYIKYKSRRSFIEEKKMP

Illustrative adenovirus E19 nucleic acid sequences (NCBI Reference Sequence: AC_000007.1 REGION: 28812 . . . 29291) are shown as SEQ ID NO: 6 and SEQ ID NO: 44.

SEQ ID NO: 6
ATGAGGTACATGATTTTAGGCTTGCTCGCCCTTGCGGCAGTCTGCAGCGCTGCCAAAAAGGTTGAGTT
TAAGGAACCAGCTTGCAATGTTACATTTAAATCAGAAGCTAATGAATGCACTACTCTTATAAAATGCA
CCACAGAACATGAAAAGCTTATTATTCGCCACAAAGACAAAATTGGCAAGTATGCTGTATATGCTATT
TGGCAGCCAGGTGACACTAACGACTATAATGTCACAGTCTTCCAAGGTGAAAATCGTAAAACTTTTAT
GTATAAATTTCCATTTTATGAAATGTGCGATATTACCATGTACATGAGCAAACAGTACAAGTIGTGGC
CCCCACAAAAGTGTTTAGAGAACACTGGCACCTTTTGTTCCACCGCTCTGCTTATTACAGCGCTTGCT
TTGGTATGTACCTTACTTTATCTCAAATACAAAAGCAGACGCAGTTTTATTGATGAAAAGAAAATGCC
TTGA
SEQ ID NO: 44
ACCCAGAAGGTGGACTTCAAAGAACCCGCCTGCAACGTGACCTTTGCCGCCGAGGCCAATGAGTGCAC
CACACTGATCAAGTGTACCACCGAGCACGAGAAGCTGCTGATCCGGCACAAGAACAAGATCGGGAAGT
ACGCCGTGTACGCCATCTGGCAGCCTGGCGATACCACAGAGTACAATGTGACCGTGTTCCAGGGCAAG
AGCCACAAGACCTTCATGTACACCTTTCCGTTCTACGAGATGTGCGACATCACGATGTACATGAGCAA
GCAGTACAAGCTGTGGCCTCCTCAGAACTGCGTCGAGAATACCGGCACCTTCTGCTGCACCGCCATGC
TGATTACAGTGCTGGCCCTCGTGTGCACCCTGCTGTACATCAAGTACAAGAGCCGGCGGAGCTTCATC
GAAGAGAAGAAGATGCCC

An illustrative HCMV US3 amino acid sequence (NCBI Reference Sequence: YP_081590.1) is shown as SEQ ID NO: 7.

SEQ ID NO: 7
MKPVLVLAILAVLELRLADSVPRPLNVVVSEIKSAHFRVEENQCWFHMGM
LYFKGRMSGNFTKKHEVNVGIVSQSYMDRLQVSGEQYHHDERGAYFEWNI
GGYPVSHTVDMVDITLSTRWGDPKKYAACVPQVRMDYSSQTINWYLQRSM
RDDNWGLLFRTLLVYLFSLVVLVLLTVGVSARLRFI

An illustrative HCMV US3 nucleic acid sequence (NCBI Reference Sequence: NC_006273.2:c200905-200345) is shown as SEQ ID NO: 8.

SEQ ID NO: 8
ATGAAGCCGGTGTTGGTGCTCGCGATCCTGGCCGTCCTGTTCCTGAGACTCGCGGACTCGGTGCCCCG
GCCCCTGAATGTGGTGGTATCGGAGATCAAATCGGCCCACTTTCGGGTGGAGGAGAACCAATGCTGGT
TCCATATGGGCATGCTGTACTTCAAGGGGAGGATGTCGGGCAACTTCACCAAGAAACACTTTGTGAAC
GTGGGTATCGTCTCTCAATCTTACATGGACAGATTGCAAGTGTCCGGTGAGCAGTACCACCATGATGA
GCGCGGAGCCTATTTCGAGTGGAACATCGGTGGGTACCCGGTGTCGCACACCGTGGATATGGTGGACA
TCACGTTGTCTACGAGGTGGGGAGACCCCAAGAAATATGCCGCGTGTGTTCCCCAGGTGAGGATGGAC
TATAGCTCTCAGACCATCAACTGGTACCTTCAGCGAAGCATGAGAGACGACAATTGGGGTCTGCTGTT
CAGAACCCTGCTCGTATATCTGTTCTCTCTGGTTGTTCTTGTGCTCCTGACCGTAGGGGTGAGCGCCC
GTCTGCGATTTATTTAA

An illustrative HCMV US10 amino acid sequence (Uniprot P09728-1) is shown as SEQ ID NO: 9.

Amino acids 1-24 of SEQ ID NO: 9 provide a signal peptide. Suitably the US10 amino acid sequence for use in the present invention may lack amino acids 1-24 of SEQ ID NO: 9.

SEQ ID NO: 9
MLRRGSLRNPLAICLLWWLGVVAAATEETREPTYFTCGCVIQNHVLKGAV
KLYGQFPSPKTLRASAWLHDGENHERHRQPILVEGTATATEALYILLPTE
LSSPEGNRPRNYSATLTLASRDCYERFVCPVYDSGTPMGVLMNLTYLWYL
GDYGAILKIYFGLFCGACVITRSLLLICGYYPPRE

An illustrative HCMV US10 nucleic acid sequence (NCBI Reference Sequence: YP_081595.1) is shown as SEQ ID NO: 10.

SEQ ID NO: 10
ATGCTACGCCGGGGAAGCCTCCGGAACCCTCTGGCGATCTGCCTGTTGTGGTGGCTGGGAGTGGTGGC
GGCAGCCACGGAGGAGACGAGAGAACCTACCTACTTCACGTGCGGCTGTGTTATTCAGAACCATGTAC
TGAAGGGTGCGGTGAAACTCTATGGACAATTCCCCTCGCCTAAGACTTTGCGGGCCTCGGCTTGGCTA
CACGACGGGGAAAATCACGAAAGGCACCGGCAGCCCATCCTCGTGGAAGGCACCGCGACAGCTACCGA
GGCGCTGTACATTCTGCTGCCCACGGAACTGTCGTCGCCGGAAGGAAACCGACCCCGAAACTACTCTG
CTACCCTAACACTCGCCTCCCGGGATTGTTATGAACGCTTCGTGTGCCCGGTATACGATTCCGGGACG
CCGATGGGCGTTTTGATGAACTTAACGTACCTCTGGTATCTAGGCGACTATGGGGCAATACTAAAAAT
TTATTTCGGACTGTTCTGCGGGGCCTGCGTCATCACCCGATCCCTCCTCCTGATATGCGGTTATTATC
CACCTCGCGAATAA

An illustrative HHV-7 U21 amino acid sequence (NCBI Reference Sequence: YP_073761.1) is shown as SEQ ID NO: 11.

SEQ ID NO: 11
MWTILLFCVPVIYGELYPDFCPLAVVDEDVNATVDDLLLEDISLSKQCSD
DKIRHSAVAAMTDNAFFFGNSETQIETDFGKYLAFNCYQVESTLNHELFK
NFKKTKGLMKRYDKLCLDVESYIHIQIICSPFKSFIRLRRMNETGISPRI
LETTFYLQNKRNSTWVAIKNYLGEDDPFTYRIWHTLTHAKNELINSCEND
ENQLFFWQRKYLSLAKTFEATFKQGENPMIEQRNEQRYRTNNIDCSFSKF
RQNGVKVAVCKYTGWGVSGFGSLEVLQKIKSPFGEEWKRVGENSTGAFTP
LYGSDVLWGLIFLRVEMTTYVCTCTNKNTGTQIQVTLPDVDLDLLDSEKT
SSNVFVDMLCYTLIAILFLAFVTAVVLLGVSCLDGVQKVLTWPLQHIQKE
PVSEKIINLTNLMFGQEPLPKKESLKQQCL

An illustrative HHV-7 U21 nucleic acid sequence (NCBI Reference Sequence: NC_001716.2:c33714-31242) is shown as SEQ ID NO: 12.

SEQ ID NO: 12
ATGTGGACTATCTTGCTGTTTTGCGTTCCTGTGATTTATGGAGAACTCTATCCGGATTTCTGTCCCTT
AGCAGTTGTGGATTTTGATGTGAATGCTACAGTGGATGATTTGCTATTGTTCGACATATCTTTGAGCA
AACAATGTTCAGACGATAAAATCAGACACTCTGCAGTCGCGGCAATGACAGACAATGCTTTCTTTTTT
GGCAACAGCGAAACGCAAATTGAAACCGATTTTGGCAAATATTTGGCTTTCAACTGTTATCAAGTATT
TTCTACACTAAATCATTTTTTGTTTAAGAACTTTAAAAAAACAAAAGGACTGATGAAGCGATATGATA
AACTCTGTCTTGATGTCGAGTCTTACATTCATATTCAAATTATCTGTTCACCCTTTAAAAGTTTTATC
CGTCTGAGACGTATGAATGAAACTGGAATTTCTCCTAGAATTCTTGAAACCACATTCTACTTGCAAAA
TAAAAGAAACAGCACGTGGGTAGCGATTAAAAACTATTTAGGTGAAGATGATCCCTTCACTTATCGAA
TTTGGCACACTTTGACACACGCTAAAAACTTTCTGATCAATTCTTGTGAAAATGACTTCAATCAACTT
TTTTTTTGGCAAAGAAAGTACCTATCACTCGCGAAAACATTCGAAGCTACATTTAAGCAAGGCTTTAA
TCCTATGATTGAACAAAGAAATGAACAACGATATAGAACTAATAACATAGATTGTTCATTTTCGAAAT
TTAGACAGAATGGTGTAAAGGTAGCAGTTTGTAAATATACAGGATGGGGTGTTAGCGGTTTCGGCTCC
TTAGAAGTTCTGCAAAAGATAAAAAGTCCGTTTGGAGAAGAGTGGAAAAGAGTTGGTTTCAACTCTAC
GGGTGCTTTTACTCCTTTGTATGGTAGTGATGTGCTTTGGGGTCTAATTTTTCTAAGAGTAGAAATGA
CCACATATGTTTGTACCTGTACGAATAAGAACACAGGAACACAAATACAGGTAACATTACCTGATGTG
GATTTGGATCTATTAGATAGTGAAAAAACGTCTAGTAATGTTTTCGTAGACATGCTTTGCTACACATT
GATTGCTATATTATTCCTTGCATTTGTTACAGCGGTAGTTTTATTAGGAGTTTCCTGTTTAGACGGTG
TTCAGAAAGTTCTGACGTGGCCTTTACAGCACATACAAAAAGAACCAGTATCAGAGAAAATAATCAAT
CTAACAAACTTGATGTTTGGACAAGAACCGTTACCTAAAAAAGAATCTCTCAAACAGCAATGTTTGTG
A

An illustrative HIV-1 Nef amino acid sequence (NCBI Reference Sequence: NP_057857.2) is shown as SEQ ID NO: 13.

SEQ ID NO: 13
MGGKWSKSSVIGWPTVRERMRRAEPAADRVGAASRDLEKHGAITSSNTAA
TNAACAWLEAQEEEEVGFPVTPQVPLRPMTYKAAVDLSHELKEKGGLEGL
IHSQRRQDILDLWIYHTQGYFPDWQNYTPGPGVRYPLTFGWCYKLVPVEP
DKIEEANKGENTSLLHPVSLHGMDDPEREVLEWREDSRLAFHHVARELHP
EYEKNC

An illustrative HIV-1 Nef nucleic acid sequence (NCBI Reference Sequence: NC_001802.1:c8343-8963) is shown as SEQ ID NO: 14.

SEQ ID NO: 14
ATGGGTGGCAAGTGGTCAAAAAGTAGTGTGATTGGATGGCCTACTGTAAGGGAAAGAATGAGACGAGC
TGAGCCAGCAGCAGATAGGGTGGGAGCAGCATCTCGAGACCTGGAAAAACATGGAGCAATCACAAGTA
GCAATACAGCAGCTACCAATGCTGCTTGTGCCTGGCTAGAAGCACAAGAGGAGGAGGAGGTGGGTTTT
CCAGTCACACCTCAGGTACCTTTAAGACCAATGACTTACAAGGCAGCTGTAGATCTTAGCCACTTTTT
AAAAGAAAAGGGGGGACTGGAAGGGCTAATTCACTCCCAAAGAAGACAAGATATCCTTGATCTGTGGA
TCTACCACACACAAGGCTACTTCCCTGATTAGCAGAACTACACACCAGGGCCAGGGGTCAGATATCCA
CTGACCTTTGGATGGTGCTACAAGCTAGTACCAGTTGAGCCAGATAAGATAGAAGAGGCCAATAAAGG
AGAGAACACCAGCTTGTTACACCCTGTGAGCCTGCATGGGATGGATGACCCGGAGAGAGAAGTGTTAG
AGTGGAGGTTTGACAGCCGCCTAGCATTTCATCACGTGGCCCGAGAGCTGCATCCGGAGTACTTCAAG
AACTGCTGA

An illustrative KSHV K3 amino acid sequence (NCBI Reference Sequence: YP_001129360.1) is shown as SEQ ID NO: 15.

SEQ ID NO: 15
MEDEDVPVCWICNEELGNERFRACGCTGELENVHRSCLSTWLTISRNTAC
QICGVVYNTRVVWRPLREMTLLPRLTYQEGLELIVFIFIMTLGAAGLAAA
TWVWLYIVGGHDPEIDHVAAAAYYVFFVFYQLFVVEGLGAFFHMMRHVGR
AYAAVNTRVEVFPYRPRPTSPECAVEEIELQEILPRGDNQDEEGPAGAAP
GDQDGPADGAPVHRDSEESVDEAAGYKEAGEPTHNDGRDDNVEPTAVGCD
CNNLGAERYRATYCGGYVGAQSGDGAYSVSCHNKAGPSSLVDILPQGLLG
GGYGSMGVIRKRSAVSSALMFH

An illustrative KSHV K3 nucleic acid sequence (NCBI Reference Sequence: NC_009333.1:c19542-18573) is shown as SEQ ID NO: 16.

SEQ ID NO: 16
ATGGAAGATGAGGATGTTCCTGTCTGCTGGATTTGCAACGAGGAGCTCGGAAATGAGAGATTTAGAGC
CTGTGGATGCACAGGAGAGCTCGAGAACGTCCATAGAAGTTGTTTAAGCACCTGGCTCACTATCTCTA
GAAACACGGCCTGTCAGATATGTGGCGTCGTATACAACACGCGCGTGGTCTGGCGACCCTTGCGCGAA
ATGACGCTATTGCCTCGGCTGACTTACCAGGAGGGTCTGGAACTGATTGTTTTTATTTTCATCATGAC
ATTGGGGGCCGCTGGCCTTGCCGCTGCGACGTGGGTTTGGCTATATATAGTGGGCGGTCATGACCCAG
AGATAGATCACGTCGCTGCAGCCGCGTACTACGTTTTTTTCGTGTTTTACCAATTGTTTGTCGTTTTT
GGGTTGGGTGCGTTTTTCCACATGATGCGCCACGTGGGGGGGGCATACGCTGCTGTAAACACCCGGGT
TGAAGTGTTTCCATATAGACCTCGGCCGACATCACCAGAGTGTGCGGTAGAGGAGATCGAGCTTCAGG
AAATTCTTCCCCGTGGGGATAACCAGGACGAGGAGGGGCCCGCGGGGGCAGCTCCAGGCGACCAAGAT
GGCCCCGCGGATGGCGCTCCTGTGCATCGCGACTCAGAAGAATCCGTGGATGAAGCTGCAGGGTACAA
GGAAGCGGGAGAACCAACACATAATGATGGACGTGATGACAATGTAGAGCCAACCGCGGTTGGGTGTG
ACTGTAACAACTTGGGCGCTGAGCGGTATAGGGCCACTTACTGTGGCGGTTATGTTGGTGCCCAGTCG
GGCGATGGGGCTTATAGTGTCTCCTGCCATAATAAGGCTGGACCCTCCTCTCTAGTTGATATCCTTCC
ACAGGGTTTGCTTGGGGGTGGCTATGGTTCCATGGGCGTGATTAGGAAACGTTCGGCTGTTTCGTCTG
CCCTTATGTTTCATTAAA

An illustrative KSHV K5 amino acid sequence (NCBI Reference Sequence: YP_001129365.1) is shown as SEQ ID NO: 17.

SEQ ID NO: 17
MASKDVEEGVEGPICWICREEVGNEGIHPCACTGELDVVHPQCLSTWLTV
SRNTACQMCRVIYRTRTQWRSRLNLWPEMERQEIFELFLLMSVVVAGLVG
VALCTWTLLVILTAPAGTESPGAVLGFLCFFGFYQIFIVEAFGGICRVSG
TVRALYAANNTRVTVLPYRRPRRPTANEDNIELTVLVGPAGGTDEEPTDE
SSEGDVASGDKERDGSSGDEPDGGPNDRAGLRGTARTDLCAPTKKPVRKN
HPKNNG

An illustrative KSHV K5 nucleic acid sequence (NCBI Reference Sequence: NC_009333.1:c26635-25865) is shown as SEQ ID NO: 18.

SEQ ID NO: 18
ATGGCGTCCAAGGACGTAGAAGAGGGTGTAGAGGGACCCATCTGCTGGATATGTAGGGAAGAGGTGGG
GAACGAGGGCATACACCCCTGCGCCTGTACCGGAGAGCTGGATGTCGTCCACCCGCAGTGTTTAAGCA
CTTGGCTAACAGTGTCTCGAAACACGGCCTGTCAAATGTGTCGCGTTATATACCGCACGCGCACGCAG
TGGCGTAGTCGCCTTAACCTGTGGCCGGAGATGGAGCGCCAAGAAATTTTTGAACTGTTTTTGCTGAT
GTCTGTGGTGGTGGCTGGGCTTGTCGGCGTGGCGTTGTGCACCTGGACGCTCCTGGTCATCCTAACTG
CTCCTGCGGGAACATTCTCCCCCGGGGCCGTGCTCGGTTTTCTCTGCTTCTTTGGGTTTTACCAAATA
TTTATTGTGTTTGCATTTGGCGGCATATGCCGCGTAAGTGGCACTGTGAGGGCATTATACGCGGCAAA
TAACACCCGGGTGACCGTACTGCCATACCGACGGCCGCGCCGGCCAACCGCGAACGAAGATAACATCG
AATTGACGGTCCTTGTCGGACCCGCGGGCGGGACGGACGAGGAGCCCACGGACGAGTCATCTGAAGGA
GACGTCGCCTCTGGAGACAAAGAACGTGACGGTTCATCCGGAGACGAGCCGGACGGGGGCCCGAACGA
CCGTGCGGGACTTAGGGGGACAGCGCGTACCGACCTATGCGCGCCCACAAAAAAGCCGGTGCGGAAAA
ATCATCCAAAAAACAACGGTTGA

The first domain may be any viral protein or a functional fragment of any viral protein which downregulates cell surface expression of MHC class I. Suitably, the first domain may be any of the viral proteins or a functional fragment of any of the viral proteins mentioned above which downregulates cell surface expression of MHC class I.

Accordingly, in a preferred embodiment, the first domain is a viral protein, or a functional fragment thereof, which is capable of downregulating cell surface expression of MHC class I molecule or a functional fragment thereof.

Preferably, the first domain may be any viral MHC class I-binding protein (e.g. a viral HLA class I-binding protein) which is capable of downregulating cell surface expression of MHC class I molecule or a functional fragment thereof. Suitable methods for determining binding will be known to those of skill in the art. For example, binding can be assessed by flow cytometry, immunohistochemistry, Western blotting, ELISA and surface plasmon resonance. It is within the ambit of the skilled person to select and implement a suitable assay to determine if a candidate domain (e.g. a peptide, antibody or an antibody-derived binding domain) is capable of binding to MHC class I.

In one embodiment, the first domain is a viral protein, or a functional fragment thereof, which induces MHC class I translocation from the ER to the cytosol and subsequent MHC class I degradation by the proteasome. Suitably, the first domain is selected from HCMV US11, HCMV US2, and/or a functional fragment thereof.

In another embodiment, the first domain is a viral protein, or a functional fragment thereof, which prevents transport of the MHC class I molecule from the ER to the plasma membrane, for example by retention in the ER or diversion to the lysosome. The first domain may be a viral protein, or a functional fragment thereof, which leads to a retention of MHC class I within the ER, Golgi apparatus and/or TGN. The first domain may be a viral protein, or a functional fragment thereof, which leads to a retention of MHC class I within the ER. The first domain may be a viral protein, or a functional fragment thereof, which retains MHC class I within the ER, Golgi apparatus and/or TGN. The first domain may be a viral protein, or a functional fragment thereof, which leads to diversion of or diverts MHC class Ito the lysosome. Suitably, the first domain is selected from, adenovirus E19, HCMV US3, HCMV US10, HHV-7 U21, or a functional fragment thereof.

In a further embodiment, the first domain is a viral protein, or a functional fragment thereof, which induces internalisation of the MHC class I molecule via endocytosis and subsequent degradation or sequestration of the MHC class I molecule. Suitably, the first domain is selected from selected from HIV-1 Nef, KSHV K3, KSHV K5, or a functional fragment thereof.

As used herein, the term “capable of downregulating cell surface expression of an MHC class I molecule” means that expression of the first domain in a cell leads to decreased cell surface expression of MHC class I compared to a corresponding cell that does not express the first domain. Thus, a cell which expresses the polypeptide of the invention has decreased cell surface expression of MHC class I compared to a corresponding cell that does not express the polypeptide of the invention. Cell surface expression of a molecule may be assessed using methods known in the art. For example, primary T-cells from a healthy donor may be transduced with a retroviral vector expressing the candidate domain or candidate polypeptide comprising the candidate domain followed by staining for expression of MHC class I and measurement e.g. by FACS (see e.g. present Example 1 and Example 2).

As used herein, the term “functional fragment” means a polypeptide or amino acid sequence which is a portion of a full-length polypeptide and which retains the function of the full-length polypeptide. For example, a functional fragment of a first domain as described herein is capable of downregulating cell surface expression of an MHC class I molecule.

When expressed in a cell, the first domain may decrease cell surface expression of MHC class I by at least 10% (suitably at least 20%, at least 30%, at least 40%, at least 50%, at least 60%, at least 70%, at least 80% or at least 90%) compared to a corresponding cell that does not express the first domain. Thus, when expressed in a cell, the polypeptide of the invention may decrease cell surface expression of MHC class I by at least 10% (suitably at least 20%, at least 30%, at least 40%, at least 50%, at least 60%, at least 70%, at least 80% or at least 90%) when expressed in a cell compared to a corresponding cell that does not express the polypeptide of the invention.

In one embodiment, the first domain is selected from HCMV US11, HCMV US2, adenovirus E19, HCMV US3, HCMV US10, HHV-7 U21, HIV-1 Nef, KSHV K3, KSHV K5, or a functional fragment thereof.

In one embodiment, the first domain is selected from HCMV US11, HCMV US2, adenovirus E19, HHV-7 U21, HIV-1 Nef, KSHV K3, KSHV K5, or a functional fragment thereof.

In one embodiment, the first domain is selected from HCMV US11, HCMV US2 or a functional fragment thereof.

In one embodiment, the first domain is selected from adenovirus E19, HCMV US3, HCMV US10, HHV-7 U21, and/or a functional fragment thereof.

In one embodiment, the first domain is selected from HIV-1 Nef, KSHV K3, KSHV K5, and/or a functional fragment thereof.

In a preferred embodiment, the first domain is HCMV US11 or HCMV US2, or a functional fragment thereof. Preferably, the first domain is HCMV US11 or a functional fragment thereof.

In one embodiment, the first domain is HCMV US2 or a functional fragment thereof.

In one embodiment, the first domain is adenovirus E19 or a functional fragment thereof.

In one embodiment, the first domain is HCMV US3 or a functional fragment thereof.

In one embodiment, the first domain is HCMV US10 or a functional fragment thereof.

In one embodiment, the first domain is HHV-7 U21 or a functional fragment thereof.

In one embodiment, the first domain is HIV-1 Nef or a functional fragment thereof.

In one embodiment, the first domain is KSHV K3 or a functional fragment thereof.

In one embodiment, the first domain is KSHV K5 or a functional fragment thereof.

In one embodiment, the first domain comprises a sequence as set forth in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 43 or a sequence having at least 80% (suitably, at least 85%, at least 90%, at least 95%, at least 96%, at least 97%, at least 98% or at least 99%) identity thereto or a functional fragment thereof.

In one embodiment, the first domain consists of a sequence as set forth in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 43 or a sequence having at least 80% (suitably, at least 85%, at least 90%, at least 95%, at least 96%, at least 97%, at least 98% or at least 99%) identity thereto or a functional fragment thereof.

In one embodiment, the first domain comprises a sequence as set forth in SEQ ID NO: 1, SEQ ID NO: 3, SEQ ID NO: 5, SEQ ID NO: 11, SEQ ID NO: 13, SEQ ID NO: 15, SEQ ID NO: 17, SEQ ID NO: 43 or a sequence having at least 80% (suitably, at least 85%, at least 90%, at st 95%, at least 96%, at least 97%, at least 98% or at least 99%) identity thereto or a functional fragment thereof.

In a preferred embodiment, the first domain comprises a sequence as set forth in SEQ ID NO: 1 or SEQ ID NO: 3, or a sequence having at least 80% (suitably, at least 85%, at least 90%, at least 95%, at least 96%, at least 97%, at least 98% or at least 99%) identity thereto or a functional fragment thereof. Preferably, the first domain comprises a sequence as set forth in SEQ ID NO: 1, or a sequence having at least 80% (suitably, at least 85%, at least 90%, at least 95%, at least 96%, at least 97%, at least 98% or at least 99%) identity thereto or a functional fragment thereof.

In one embodiment, the first domain comprises a sequence as set forth in SEQ ID NO: 3, or a sequence having at least 80% (suitably, at least 85%, at least 90%, at least 95%, at least 96%, at least 97%, at least 98% or at least 99%) identity thereto or a functional fragment thereof.

In one embodiment, the first domain comprises a sequence as set forth in SEQ ID NO: 5, or a sequence having at least 80% (suitably, at least 85%, at least 90%, at least 95%, at least 96%, at least 97%, at least 98% or at least 99%) identity thereto or a functional fragment thereof.

In one embodiment, the first domain comprises a sequence as set forth in SEQ ID NO: 7, or a sequence having at least 80% (suitably, at least 85%, at least 90%, at least 95%, at least 96%, at least 97%, at least 98% or at least 99%) identity thereto or a functional fragment thereof.

In one embodiment, the first domain comprises a sequence as set forth in SEQ ID NO: 9, or a sequence having at least 80% (suitably, at least 85%, at least 90%, at least 95%, at least 96%, at least 97%, at least 98% or at least 99%) identity thereto or a functional fragment thereof.

In one embodiment, the first domain comprises a sequence as set forth in SEQ ID NO: 11 or a sequence having at least 80% (suitably, at least 85%, at least 90%, at least 95%, at least 96%, at least 97%, at least 98% or at least 99%) identity thereto or a functional fragment thereof.

In one embodiment, the first domain comprises a sequence as set forth in SEQ ID NO: 13, or a sequence having at least 80% (suitably, at least 85%, at least 90%, at least 95%, at least 96%, at least 97%, at least 98% or at least 99%) identity thereto or a functional fragment thereof.

In one embodiment, the first domain comprises a sequence as set forth in SEQ ID NO: 15, or a sequence having at least 80% (suitably, at least 85%, at least 90%, at least 95%, at least 96%, at least 97%, at least 98% or at least 99%) identity thereto or a functional fragment thereof.

In one embodiment, the first domain comprises a sequence as set forth in SEQ ID NO: 17, or a sequence having at least 80% (suitably, at least 85%, at least 90%, at least 95%, at least 96%, at least 97%, at least 98% or at least 99%) identity thereto or a functional fragment thereof.

In one embodiment, the first domain comprises a sequence as set forth in SEQ ID NO: 43, or a sequence having at least 80% (suitably, at least 85%, at least 90%, at least 95%, at least 96%, at least 97%, at least 98% or at least 99%) identity thereto or a functional fragment thereof.

### Second Domain

The target molecule can be any protein which it is desired to prevent or reduce cell surface expression of. For example, the target molecule may be a component of the TCR/CD3 complex, MHC class II, co-receptors or co-inhibitory receptors. Most co-receptor (including inhibitory co-receptor) molecules are members of the immunoglobulin superfamily (IgSF) and tumour necrosis factor receptor superfamily (TNFRSF). Suitably, the co-receptor may be cluster of differentiation (CD) 4, CD8, CD28, inducible T-cell costimulator (ICOS), CD226, CD355, T-cell immunoglobulin and mucin domain 1 (TIM1), CD2, signalling lymphocytic activation molecule (SLAM), CD244, CD352, CD84, CD229, CD319, CD137, CD134, CD27, CD357, CD30, CD270, lymphotoxin β receptor (LTβR) or death receptor 3 (DR3). Suitably, the inhibitory co-receptor may be cytotoxic T lymphocyte antigen 4 (CTLA-4), programmed cell death protein 1 (PD-1), programmed cell death protein 1 homolog (PD1H), CD272, CD80, CD274, T-cell immunoreceptor with Ig and ITIM domains (TIGIT), TIM2, TIM3, CD244, CD352, Leukocyte-associated immunoglobulin-like receptor 1 (LAIR1), CD223, CD160 or CD358.

MHC class II molecules are present in antigen-presenting cells such as dendritic cells, mononuclear phagocytes, some endothelial cells, thymic epithelial cells, and B cells. MHC class II molecules present on the cell surface peptide fragments derived from breakdown of extracellular proteins (not intracellular proteins as in MHC class I) processed by the host cellular machinery. The HLAs corresponding to MHC class II are HLA-DP, HLA-DM, HLA-DOA, HLA-DOB, HLA-DQ, and HLA-DR.

MHC class II molecules are heterodimers of an α chain and a β chain and undergo folding and assembly within the ER lumen. During synthesis in the ER, the α and β chains are produced and complexed with the invariant chain, which prevents the peptide binding cleft of the MHC class II molecule from binding intracellular peptides. MHC class II molecules are exported from the ER to the Golgi, followed by fusion with a late endosome containing endocytosed, degraded proteins. The MHC Class II molecules then bind extracellular peptide fragments and are presented on the cell surface.

As used herein, the terms “MHC class II molecule” and “MHC class II” are used interchangeably to refer to heterodimers of an α chain and a β chain which are encoded by the MHC locus.

The TCR/CD3 complex, also termed the TCR complex, is a multimeric complex on the T-cell surface whose activation leads to the activation of the T-cell. The complex comprises (i) TCR, (ii) CD3 T-cell co-receptor. Both TCRs and CD3 are required for the antigen-specific activation of T-cells. Every T-cell expresses clonal TCRs which recognize specific peptide/MHC complexes during physical contact between T-cell and antigen-presenting cell (via MHC class II) or any other cell type (via MHC class I). The TCR is a disulfide-linked membrane-localised heterodimeric protein comprising the highly variable α and β chains. CD3 links antigen recognition by the TCR with intracellular signalling events downstream of the TCR T3 zeta-chain. CD3 is a protein complex comprising six distinct chains. In mammals, CD3 comprises a CD3γ chain, a CD3δ chain, two CD3ε chains and two CD3ζ accessory molecules.

Accordingly, the TCR/CD3 complex typically comprises several components: TCR α-chain, TCR β-chain, CD3γ chain, CD3δ chain, two CD3ε chains, and two CD3ζ chains. The second domain may bind to one or more of these components. Suitably, the second domain may be capable of binding to two or more, three or more, four or more, five or more, or six or more of these components. The one or more components to which the second domain is capable of binding may be assembled to form the TCR/CD3 complex. Thus, the one or more components may be part of the TCR/CD3 complex. Preferably, the one or more components are not assembled to form the TCR/CD3 complex. Thus, the second domain may be capable of binding to a single TCR/CD3 component that is not associated with any of the other components or to one or more components that are associated with each other but which do not form the full TCR/CD3 complex. Hence, the second domain may be capable of binding to a nascent, incomplete form of the TCR/CD3 complex.

T-cell receptor assembly is reviewed by Call and Wucherpfennig, 2005 (Call, M. E. & Wucherpfennig, K. W. (2005) Annu. Rev. Immunol. 23: 101-125). Briefly, The TCR complex is composed of TCRα, TCRβ, CD3γ, ζ, δ and ε. Assembly results in alignment of polar residues in the transmembrane domain. Each assembly step thus results in the formation of a three-helix interface in the membrane that involves one basic and two acidic TM residues, and this arrangement effectively shields these ionizable residues at protein—protein interfaces from the lipid. Since proteins whose TM domains have exposed ionizable residues are not stably integrated into the lipid bilayer, assembly based on shielding of ionizable residues permits full equilibration of the receptor into the lipid bilayer and prevents degradation. Assembly and export of intact receptor complexes is precisely regulated with degradation of unassembled components.

The second domain is capable of binding to a component of TCR/CD3 complex or MHC class II. In one embodiment, the second domain is capable of binding to a component of TCR/CD3 complex. In an alternative embodiment, the second domain is capable of binding to MHC class II.

Numerous binding domains are known in the art, including those based on the antigen binding site of an antibody, antibody mimetics, and TCRs. The second domain may be a binding domain known in the art. For example, the second domain may comprise: an antibody or a fragment thereof; a single-chain variable fragment (scFv) derived from a monoclonal antibody; a natural ligand of the target molecule; a peptide with sufficient affinity for the target; a single domain binder such as a camelid; an artificial binder single as a Darpin; or a single-chain derived from a TCR. The antibody or a fragment thereof may be a full-length antibody, a single chain antibody fragment, a F(ab) fragment, a F(ab′)2 fragment, a F(ab′) fragment, a single domain antibody (sdAb), a VHH/nanobody, a nanobody, an affibody, a fibronectin artificial antibody scaffold, an anticalin, an affilin, a DARPin, a VNAR, an iBody, an affimer, a fynomer, a domain antibody (DAb), an abdurin/nanoantibody, a centyrin, an alphabody, a nanofitin or a D domain. Preferably, the second domain is an antibody or a fragment thereof. Preferably, the second domain is an scFv.

The term “antibody fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Antibody fragments include Fab, Fab′, F(ab′) 2, Fabc, Fd, dAb, Fv, single chains, single-chain antibodies, e.g. scFv, single domain antibodies, and an isolated complementarity determining region (CDR).

The second domain (e.g. antibody or fragment thereof) may be non-human, chimeric, humanised or fully human.

Suitably, the ability of a candidate domain to bind the target molecule (e.g. a component of the TCR/CD3 complex or MHC class II) may be determined using an isolated (e.g. recombinant) protein. Suitable methods for determining binding will be known to those of skill in the art. For example, binding may be assessed by flow cytometry, immunohistochemistry, Western blotting, ELISA and surface plasmon resonance. It is within the ambit of the skilled person to select and implement a suitable assay to determine if a candidate domain (e.g. a peptide, antibody or an antibody-derived binding domain) is capable of binding to a given target molecule.

The generation of binding domains that recognise a target molecule can be performed using methods known in the art. Suitably, the second domain may be a binding domain which has been generated to recognise a component of the TCR/CD3 complex or MHC class II. The second domain may be an scFv or single domain binder which has been generated to recognise a component of the TCR/CD3 complex or MHC class II.

Binding domains that are capable of binding to a component of TCR/CD3 complex or MHC class II are known in the art (see, for example, Routledge et al. (1991) Eur. J. Immunol. 21: 2717-2725; Elsasser et al. (1996) Blood 87: 3803-3812; Bolt et al. (1993) Eur. J. Immunol. 23: 403-411; and Traunecker et al. (1991) EMBO J. 10: 3655-3659). The selection of a suitable second domain for use in the polypeptide of the invention is within the capabilities of the skilled person.

Suitably, the binding domain binds to one or more of the components of the TCR/CD3 complex, wherein the one or more components are not assembled to form the TCR/CD3 complex. In other words, binding may occur to a single component that is not associated with any of the other components, reflecting an ability to bind the nascent assembling TCR/CD3 complex before access to the ER or Golgi has passed. Suitably, the binding domain may be suitable for intracellular staining of one or more components on the TCR/CD3 complex. Without wishing to be bound by theory, it is considered that utility in intracellular staining may correlate with the ability of the binding domain to recognise the nascent assembling TCR/CD3 complex.

Suitably, the second binding domain is not a OKT3 antibody or derived from a OKT3 antibody. Suitably, the second binding domain is not a BMA031 or derived from a BMA031 antibody.

The second domain preferably binds to CD3c. More preferably, the second domain is a UCHT1 antibody, or is derived from a UCHT1 antibody. Thus, the second domain may be produced by or derived from a product of a UCHT1 hybridoma. For example, the second domain may be an antibody or a fragment thereof (e.g. an scFv) derived from the UCHT1 hybridoma.

An illustrative TCR/CD3 complex binding domain amino acid sequence (UCHT1_VH-linker-UCHT1_VL) is shown as SEQ ID NO: 19, with CDR sequences indicated in bold and underlined.

SEQ ID NO: 19
DIQMTQSPSSLSASVGNRVTITCRASQDIRNYLNWYQQKPGKAPKLLIYY
TSRLESGVPSRFSGSGSGTDYTLTISSLQPEDFATYYCQQGNTLPWTFGQ
GTKVEIKSGGGGSGGGGSGGGGSEVQLVESGGGLVQPGGSLRLSCAASGY
SFTGYTMNWVRQAPGKGLEWVALINPYKGVSTYNQKFKDRFTISVDKSKN
TAYLQMNSLRAEDTAVYYCARSGYYGDSDWYFDVWGQGTLVTVS

An illustrative TCR/CD3 complex binding domain nucleic acid sequence (UCHT1_VH-linker-UCHT1_VL) is shown as SEQ ID NO: 20.

SEQ ID NO: 20
ATGGAAACCGACACCCTGCTGCTGTGGGTGCTGCTCGTGTGGATTCCTGGCAGCACCGGCGACATCCA
GATGACCCAGAGCCCTAGCAGCCTGAGCGCCAGCGTGGGCAACAGAGTGACCATCACCTGTCGGGCCA
GCCAGGACATCCGGAACTACCTGAACTGGTATCAGCAGAAGCCCGGCAAGGCCCCCAAGCTGCTGATC
TACTACACCAGCAGACTGGAAAGCGGCGTGCCCAGCAGATTTTCCGGCAGCGGCTCTGGCACCGACTA
CACCCTGACAATCAGCAGCCTGCAGCCCGAGGACTTCGCCACCTACTATTGCCAGCAGGGCAACACCC
TGCCCTGGACCTTTGGCCAGGGCACCAAGGTGGAAATCAAGAGCGGCGGAGGCGGATCTGGCGGAGGG
GGAAGTGGCGGGGGAGGATCTGAAGTGCAGCTGGTGGAATCTGGCGGCGGACTGGTGCAGCCTGGCGG
ATCTCTGAGACTGAGCTGTGCCGCCAGCGGCTACAGCTTCACCGGCTACACCATGAACTGGGTGCGCC
AGGCCCCTGGCAAGGGACTGGAATGGGTGGCCCTGATCAACCCCTACAAGGGCGTGTCCACCTACAAC
CAGAAGTTCAAGGATCGGTTCACCATCAGCGTGGACAAGAGCAAGAACACCGCCTACCTGCAGATGAA
CAGCCTGAGAGCCGAGGACACCGCCGTGTACTACTGTGCCAGAAGCGGCTACTACGGCGACAGCGACT
GGTACTTCGACGTGTGGGGCCAGGGAACCCTCGTGACCGTGTCTAGC

A further illustrative TCR/CD3 complex binding domain amino acid sequence (anti-TCR YTH clone; Bolt et al. (1993) Eur. J. Immunol. 23: 403-411) is shown as SEQ ID NO: 21, with CDR sequences indicated in bold and underlined.

SEQ ID NO: 21
DFMLTQPHSVSESPGKTVIISCTLSSGNIENNYVHWYQQRPGRAPTTVIF
DDDKRPDGVPDRFSGSIDRSSNSASLTISGLQTEDEADYYCHSYVSSFNV
FGGGTKLTVLSGGGGSGGGGSGGGGSEVQLLESGGGLVQPGGSLRLSCAA
SGFTFSSFPMAWVRQAPGKGLEWVSTISTSGGRTYYRDSVKGRFTISRDN
SKNTLYLQMNSLRAEDTAVYYCAKFRQYSGGFDYWGQGTLVTVS

A further illustrative TCR/CD3 complex binding domain nucleic acid sequence (anti-TCR YTH clone; Bolt et al. (1993) Eur. J. Immunol. 23: 403-411) is shown as SEQ ID NO: 22.

SEQ ID NO: 22
ATGGAGACCGACACCCTGCTGCTGTGGGTGCTGCTGCTGTGGGTGCCAGG
CAGCACCGGCGACTTCATGCTGACCCAGCCCCACAGCGTGAGCGAGAGCC
CAGGCAAGACCGTGATCATCAGCTGCACCCTGAGCAGCGGCAACATCGAG
AACAACTACGTGCACTGGTACCAGCAGCGGCCAGGCCGGGCCCCAACCAC
CGTGATCTTCGACGACGACAAGCGGCCCGACGGCGTGCCCGACCGCTTCA
GCGGCAGCATCGACCGGAGCAGCAACAGCGCCAGCCTGACCATCAGCGGC
CTGCAGACCGAGGACGAGGCCGACTACTACTGCCACAGCTACGTGAGCAG
CTTCAACGTGTTCGGCGGCGGCACCAAGCTGACCGTGCTGAGCGGTGGCG
GTGGCAGCGGCGGCGGCGGAAGCGGAGGTGGTGGCAGCGAGGTGCAGCTG
CTGGAGAGCGGCGGCGGCCTGGTGCAGCCCGGCGGCAGCCTGCGGCTGAG
CTGCGCCGCCAGCGGCTTCACCTTCAGCAGCTTCCCCATGGCCTGGGTGC
GGCAGGCCCCAGGCAAGGGCCTGGAGTGGGTGAGCACCATCAGCACCAGC
GGCGGCCGGACCTACTACCGGGACAGCGTGAAGGGCCGGTTCACCATCAG
CCGGGACAACAGCAAGAACACCCTGTACCTGCAGATGAACAGCCTGCGGG
CCGAGGACACCGCCGTGTACTACTGCGCCAAGTTCCGGCAGTACAGCGGC
GGCTTCGACTACTGGGGACAGGGCACCCTGGTGACCGTGAGC

A further illustrative TCR/CD3 complex binding domain amino acid sequence (TR-66 anti-TCR; Traunecker et al. (1991) EMBO J. 10: 3655-3659) is shown as SEQ ID NO: 23, with CDR sequences indicated in bold and underlined.

SEQ ID NO: 23
DIKLQQSGAELARPGASVKMSCKTSGYTFTRYTMHWVKQRPGQGLEWIGY
INPSRGYTNYNQKFKDKATLTTDKSSSTAYMQLSSLTSEDSAVYYCARYY
DDHYCLDYWGQGTTLTVSSGGGGSGGGGSGGGGSDIQLTQSPAIMSASPG
EKVTMTCRASSSVSYMNWYQQKSGTSPKRWIYDTSKVASGVPYRFSGSGS
GTSYSLTISSMEAEDAATYYCQQWSSNPLTFGAGTKLELKR

A further illustrative TCR/CD3 complex binding domain nucleic acid sequence (TR-66 anti-TCR; Traunecker et al. (1991) EMBO J. 10: 3655-3659) is shown as SEQ ID NO: 24.

SEQ ID NO: 24
ATGGAGACCGACACCCTGCTGCTGTGGGTGCTGCTGCTGTGGGTGCCCGG
CAGCACCGGCGACATCAAGCTGCAGCAGAGCGGCGCCGAGCTGGCCCGGC
CTGGAGCCAGCGTGAAGATGAGCTGCAAGACCAGCGGCTACACCTTCACC
CGGTACACCATGCACTGGGTGAAGCAGCGGCCTGGCCAGGGCCTGGAGTG
GATCGGCTACATCAACCCCAGCCGCGGCTACACCAACTACAACCAGAAGT
TCAAGGACAAGGCCACCCTGACCACCGACAAGAGCAGCAGCACCGCCTAC
ATGCAGCTGAGCAGCCTGACCAGCGAGGACAGCGCCGTGTACTACTGCGC
CCGGTACTACGACGACCACTACTGCCTGGACTACTGGGGCCAGGGCACCA
CCCTGACCGTGAGCAGCGGTGGCGGTGGCAGCGGCGGCGGCGGAAGCGGA
GGTGGTGGCAGCGACATCCAGCTGACCCAGAGCCCCGCCATCATGAGCGC
CAGCCCTGGCGAGAAGGTGACCATGACCTGCCGGGCCAGCAGCAGCGTGA
GCTACATGAACTGGTACCAGCAGAAGAGCGGCACCAGCCCTAAGCGGTGG
ATCTACGACACCAGCAAGGTGGCCAGCGGCGTGCCCTATCGGTTCAGCGG
CAGCGGCAGCGGCACCAGCTACAGCCTGACCATCAGCAGCATGGAGGCCG
AGGACGCCGCCACCTACTACTGCCAGCAGTGGAGCAGCAATCCTCTGACC
TTCGGCGCCGGCACCAAGCTGGAGCTGAAGCGGTCG

An illustrative MHC class II complex binding domain amino acid sequence (F3.3; Elsasser et al. (1996) Blood 87: 3803-3812) is shown as SEQ ID NO: 25, with CDR sequences indicated in bold and underline.

SEQ ID NO: 25
MGWSCIILFLVATATGVHSQIVLTQSPAIMSASPGEKVTMTCSASSIVRY
MYWFQQKPGSSPRLLIYDTSSLSSGVPVRESGSGSGTSYSLTISRMEAED
AATYFCQQWSSYPLTFGGGTKLEIKRSGGGGSGGGGSGGGGSEVKLEESG
GGLVQPGGSMKLSCVASGFTFSNSWMNWVRQSPEKGLEWVAEIRLKSNNY
ATRYAESVKGRFTISRDDSKSSVYLQMNNLRSEDTAIYYCTPLSYSFDYW
GQTTVTVSTA

An illustrative MHC class II complex binding domain nucleic acid sequence (F3.3; Elsasser et al. (1996) Blood 87: 3803-3812) is shown as SEQ ID NO: 26.

SEQ ID NO: 26
ATGGGCTGGAGCTGTATCATCCTTTTTCTAGTAGCAACTGCAACCGGTGT
ACACTCTCAAATTGTTCTTACTCAGTCTCCCGCCATAATGAGCGCGTCAC
CCGGAGAGAAAGTGACAATGACTTGCTCAGCCTCATCCATAGTACGATAC
ATGTATTGGTTCCAGCAGAAACCAGGCTCATCACCGAGACTGTTGATTTA
CGATACTTCCAGTCTGTCTAGTGGTGTGCCGGTCAGATTCTCAGGGTCCG
GCTCTGGTACCTCTTATTCTCTCACCATATCACGCATGGAGGCTGAAGAC
GCTGCCACATATTTCTGCCAGCAATGGAGTTCTTACCCTCTTACGTTCGG
GGGCGGGACTAAGTTGGAAATCAAAAGATCAGGAGGGGGGGGTAGCGGAG
GTGGCGGCTCAGGAGGGGGTGGATCAGAGGTAAAACTGGAGGAAAGCGGT
GGTGGACTGGTACAACCAGGGGGCTCCATGAAGCTTTCTTGTGTTGCGAG
TGGATTCACGTTCTCAAATTCCTGGATGAACTGGGTGCGACAAAGCCCCG
AAAAGGGGCTTGAATGGGTCGCTGAAATCCGATTGAAATCCAATAACTAC
GCCACTCGGTACGCAGAGTCTGTAAAGGGTCGCTTCACGATTAGCAGGGA
TGATTCCAAAAGTTCCGTTTATTTGCAAATGAATAATCTTCGGTCTGAGG
ACACGGCGATTTATTACTGCACCCCCCTCAGTTATTCCTTTGATTATTGG
GGTCAAACAACAGTGACGGTGTCAACGGCC

Suitable, illustrative anti-CD3 VHH sequences are described in WO2018/048318. An illustrative TCR/CD3 complex binding VHH is shown as SEQ ID NO: 42.

EVQAESGGGSVQTGGSLRLSCAYTASSVCMAWFRQAPGKEREGVAVTREG
LTKTGYADSVKGRFAISQDYAKKTLYLQMSSLKPEDTARYYCAARPTSPC
TVDGELLASTYNYWGQGTQVTVSS

In one embodiment, the second domain comprises a sequence as set forth in SEQ ID NO: 19, SEQ ID NO: 21, SEQ ID NO: 23, SEQ ID NO: 25 or SEQ ID NO: 42, or a sequence having at least 80% (suitably, at least 85%, at least 90%, at least 95%, at least 96%, at least 97%, at least 98% or at least 99%) identity thereto or a functional fragment thereof.

In one embodiment, the second domain consists of a sequence as set forth in SEQ ID NO: 19, SEQ ID NO: 21, SEQ ID NO: 23, SEQ ID NO: 25 or SEQ ID NO: 42, or a sequence having at least 80% (suitably, at least 85%, at least 90%, at least 95%, at least 96%, at least 97%, at least 98% or at least 99%) identity thereto or a functional fragment thereof.

In a preferred embodiment, the second domain comprises a sequence as set forth in SEQ ID NO: 19, or a sequence having at least 80% (suitably, at least 85%, at least 90%, at least 95%, at least 96%, at least 97%, at least 98% or at least 99%) identity thereto or a functional fragment thereof.

In a further embodiment, the second domain comprises a sequence as set forth in SEQ ID NO: 42, or a sequence having at least 80% (suitably, at least 85%, at least 90%, at least 95%, at least 96%, at least 97%, at least 98% or at least 99%) identity thereto or a functional fragment thereof.

As described herein, expression of the polypeptide of the invention may achieve simultaneous downregulated cell surface expression of both the native HLA class I molecule and the native target molecule (i.e. TCR or MHC class II).

As used herein, references to expression of native TCR refer to cell surface expression of functional TCR/CD3 complex.

Thus, when expressed in a cell, the polypeptide of the invention may:
(a) decrease cell surface expression of MHC class I by at least 10% (suitably at least 20%, at least 30%, at least 40%, at least 50%, at least 60%, at least 70%, at least 80%, at least 90% or 100%); and
(b) (i) decrease cell surface expression of MHC class II by at least 10% (suitably at least 20%, at least 30%, at least 40%, at least 50%, at least 60%, at least 70%, at least 80%, at least 90% or 100%); or
   (ii) decrease cell surface expression of the native TCR by at least 10% (suitably at least 20%, at least 30%, at least 40%, at least 50%, at least 60%, at least 70%, at least 80%, at least 90% or 100%),
      compared to a corresponding cell that does not express the polypeptide of the invention.

The polypeptide of the invention may decrease cell surface expression of MHC class I and MHC class II or of MHC class I and the native TCR to a different extent for each molecule.

In one embodiment, when expressed in a cell, the polypeptide of the invention decreases (i) MHC class I cell surface expression and (ii) MHC class II or TCR cell surface expression by at least 10% (suitably at least 20%, at least 30%, at least 40%, at least 50%, at least 60%, at least 70%, at least 80%, at least 90% or 100%) compared to a corresponding cell which does not express the polypeptide of the invention.

Suitably, MHC class I cell surface expression may be decreased by at least 10% (suitably at least 20%, at least 30%, at least 40%, at least 50%, at least 60%, at least 70%, at least 80%, at least 90% or 100%) compared to a corresponding cell which does not comprise the polynucleotide of the invention, the nucleic acid construct of the invention or the vector of the invention.

Suitably, MHC class II cell surface expression may be decreased by at least 10% (suitably at least 20%, at least 30%, at least 40%, at least 50%, at least 60%, at least 70%, at least 80%, at least 90% or 100%) compared to a corresponding cell which does not comprise the polynucleotide of the invention, the nucleic acid construct of the invention or the vector of the invention.

Suitably, TCR cell surface expression may be decreased by at least 10% (suitably at least 20%, at least 30%, at least 40%, at least 50%, at least 60%, at least 70%, at least 80%, at least 90% or 100%) compared to a corresponding cell which does not comprise the polynucleotide of the invention, the nucleic acid construct of the invention or the vector of the invention.

Cell surface expression of a molecule may be assessed using methods known in the art. For example, primary T-cells from a healthy donor may be transduced with a retroviral vector expressing the candidate domain or candidate polypeptide comprising the candidate domain followed by staining for expression of the molecule of interest (e.g. TCR) and measurement e.g. by FACS (see e.g. present Example 1 and Example 2). For instance, T-cells surface-stained (i.e. stained without a permeabilisation step) with an antibody or other molecule that binds to the molecule of interest (e.g. TCR) may be analysed by flow cytometry or fluorescence microscopy. Using flow cytometry, reduction in the mean fluorescence intensity (MFI) of surface-staining of the molecule of interest (e.g. TCR) in study T-cells compared to control T-cells indicates a reduction in surface expression of the molecule of interest (e.g. native TCR). Suitably, the MFI (and thus surface expression) may be reduced by up to 100%, such as up to 99%, up to 98%, up to 95%, up to 90%, up to 85%, up to 80%, up to 75%, up to 70%, up to 60%, up to 50%, up to 40%, or up to 25%, in study T-cells compared to control T-cells.

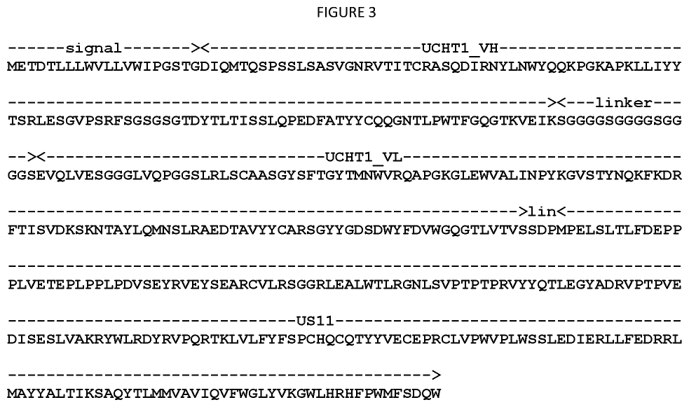

An illustrative polypeptide sequence (UCHT1-US11) is shown in FIG. 3 and as SEQ ID NO: 34.

SEQ ID NO: 34
DIQMTQSPSSLSASVGNRVTITCRASQDIRNYLNWYQQKPGKAPKLLIYY
TSRLESGVPSRFSGSGSGTDYTLTISSLQPEDFATYYCQQGNTLPWTFGQ
GTKVEIKSGGGGSGGGGSGGGGSEVQLVESGGGLVQPGGSLRLSCAASGY
SFTGYTMNWVRQAPGKGLEWVALINPYKGVSTYNQKFKDRFTISVDKSKN
TAYLQMNSLRAEDTAVYYCARSGYYGDSDWYFDVWGQGTLVTVSSDPMPE
LSLTLFDEPPPLVETEPLPPLPDVSEYRVEYSEARCVLRSGGRLEALWTL
RGNLSVPTPTPRVYYQTLEGYADRVPTPVEDISESLVAKRYWLRDYRVPQ
RTKLVLFYFSPCHQCQTYYVECEPRCLVPWVPLWSSLEDIERLLFEDRRL
MAYYALTIKSAQYTLMMVAVIQVFWGLYVKGWLHRHFPWMESDQW

An illustrative nucleic acid sequence encoding the illustrative polypeptide sequence (UCHT1-US11) above is shown as SEQ ID NO: 35.

SEQ ID NO: 35
GACATCCAGATGACCCAGAGCCCTAGCAGCCTGAGCGCCAGCGTGGGCAA
CAGAGTGACCATCACCTGTCGGGCCAGCCAGGACATCCGGAACTACCTGA
ACTGGTATCAGCAGAAGCCCGGCAAGGCCCCCAAGCTGCTGATCTACTAC
ACCAGCAGACTGGAAAGCGGCGTGCCCAGCAGATTTTCCGGCAGCGGCTC
TGGCACCGACTACACCCTGACAATCAGCAGCCTGCAGCCCGAGGACTTCG
CCACCTACTATTGCCAGCAGGGCAACACCCTGCCCTGGACCTTTGGCCAG
GGCACCAAGGTGGAAATCAAGAGCGGCGGAGGCGGATCTGGCGGAGGGGG
AAGTGGCGGGGGAGGATCTGAAGTGCAGCTGGTGGAATCTGGCGGCGGAC
TGGTGCAGCCTGGCGGATCTCTGAGACTGAGCTGTGCCGCCAGCGGCTAC
AGCTTCACCGGCTACACCATGAACTGGGTGCGCCAGGCCCCTGGCAAGGG
ACTGGAATGGGTGGCCCTGATCAACCCCTACAAGGGCGTGTCCACCTACA
ACCAGAAGTTCAAGGATCGGTTCACCATCAGCGTGGACAAGAGCAAGAAC
ACCGCCTACCTGCAGATGAACAGCCTGAGAGCCGAGGACACCGCCGTGTA
CTACTGTGCCAGAAGCGGCTACTACGGCGACAGCGACTGGTACTTCGACG
TGTGGGGCCAGGGAACCCTCGTGACCGTGTCTAGCGATCCTATGCCCGAG
CTGAGCCTGACCCTGTTCGACGAGCCACCACCCCTGGTGGAGACCGAGCC
CCTGCCACCCCTGCCCGACGTGAGCGAGTACCGGGTGGAGTACAGCGAGG
CCCGGTGCGTGCTGCGGAGCGGCGGCCGGCTGGAGGCCCTGTGGACCCTG
CGGGGCAACCTGAGCGTGCCCACCCCAACCCCACGGGTGTACTACCAGAC
CCTGGAGGGCTACGCCGACCGGGTGCCCACCCCAGTGGAGGACATCAGCG
AGAGCCTGGTGGCCAAGCGGTACTGGCTGCGGGACTACCGGGTGCCCCAG
CGGACCAAGCTGGTGCTGTTCTACTTCAGCCCCTGCCACCAGTGCCAGAC
CTACTACGTGGAGTGCGAGCCCCGGTGCCTGGTGCCCTGGGTGCCCCTGT
GGAGCAGCCTGGAGGACATCGAGCGGCTGCTGTTCGAGGACCGGCGGCTG
ATGGCCTACTACGCCCTGACCATCAAGAGCGCCCAGTACACCCTGATGAT
GGTGGCCGTGATCCAGGTGTTCTGGGGCCTGTACGTGAAGGGCTGGCTGC
ACCGGCACTTCCCCTGGATGTTCAGCGACCAGTGG

In one embodiment, the polypeptide comprises a sequence as set forth in SEQ ID NO: 34, or a sequence having at least 80% identity thereto or a functional fragment thereof.

In one embodiment, the polypeptide consists of a sequence as set forth in SEQ ID NO: 34, or a sequence having at least 80% identity thereto or a functional fragment thereof.

An illustrative polypeptide sequence (UCHT1-E19) is shown as SEQ ID NO: 40.

SEQ ID NO: 40
DIQMTQSPSSLSASVGNRVTITCRASQDIRNYLNWYQQKPGKAPKLLIYY
TSRLESGVPSRFSGSGSGTDYTLTISSLQPEDFATYYCQQGNTLPWTFGQ
GTKVEIKSGGGGSGGGGSGGGGSEVQLVESGGGLVQPGGSLRLSCAASGY
SFTGYTMNWVRQAPGKGLEWVALINPYKGVSTYNQKFKDRFTISVDKSKN
TAYLQMNSLRAEDTAVYYCARSGYYGDSDWYFDVWGQGTLVTVSSDPTQK
VDFKEPACNVTFAAEANECTTLIKCTTEHEKLLIRHKNKIGKYAVYAIWQ
PGDTTEYNVTVFQGKSHKTFMYTFPFYEMCDITMYMSKQYKLWPPQNCVE
NTGTFCCTAMLITVLALVCTLLYIKYKSRRSFIEEKKMP

In one embodiment, the polypeptide comprises a sequence as set forth in SEQ ID NO: 40, or a sequence having at least 80% (suitably, at least 85%, at least 90%, at least 95%, at least 96%, at least 97%, at least 98% or at least 99%) identity thereto or a functional fragment thereof.

An illustrative polypeptide sequence (antiCD3 VHH-US11) is shown as SEQ ID NO: 41.

SEQ ID NO: 41
EVQAESGGGSVQTGGSLRLSCAYTASSVCMAWFRQAPGKEREGVAVTREG
LTKTGYADSVKGRFAISQDYAKKTLYLQMSSLKPEDTARYYCAARPTSPC
TVDGELLASTYNYWGQGTQVTVSSDPMPELSLTLFDEPPPLVETEPLPPL
PDVSEYRVEYSEARCVLRSGGRLEALWTLRGNLSVPTPTPRVYYQTLEGY
ADRVPTPVEDISESLVAKRYWLRDYRVPQRTKLVLFYFSPCHQCQTYYVE
CEPRCLVPWVPLWSSLEDIERLLFEDRRLMAYYALTIKSAQYTLMMVAVI
QVFWGLYVKGWLHRHFPWMFSDQW

In one embodiment, the polypeptide comprises a sequence as set forth in SEQ ID NO: 41, or a sequence having at least 80% (suitably, at least 85%, at least 90%, at least 95%, at least 96%, at least 97%, at least 98% or at least 99%) identity thereto or a functional fragment thereof.

An illustrative polypeptide sequence (UCHT1-E19) is shown as SEQ ID NO: 45.

SEQ ID NO: 45
EVQAESGGGSVQTGGSLRLSCAYTASSVCMAWFRQAPGKEREGVAVTREG
LTKTGYADSVKGRFAISQDYAKKTLYLQMSSLKPEDTARYYCAARPTSPC
TVDGELLASTYNYWGQGTQVTVSDPTQKVDFKEPACNVTFAAEANECTTL
IKCTTEHEKLLIRHKNKIGKYAVYAIWQPGDTTEYNVTVFQGKSHKTFMY
TFPFYEMCDITMYMSKQYKLWPPQNCVENTGTFCCTAMLITVLALVCTLL
YIKYKSRRSFIEEKKMP

In one embodiment, the polypeptide comprises a sequence as set forth in SEQ ID NO: 45, or a sequence having at least 80% (suitably, at least 85%, at least 90%, at least 95%, at least 96%, at least 97%, at least 98% or at least 99%) identity thereto or a functional fragment thereof.

### Polynucleotide

The present invention provides a polynucleotide encoding the polypeptide as described herein.

As used herein, the terms “polynucleotide”, “nucleotide”, and “nucleic acid” are intended to be synonymous with each other.

### Nucleic Acid Construct

The present invention provides a nucleic acid construct comprising the polynucleotide as described herein.

The nucleic acid construct may further comprise a nucleic acid sequence encoding a chimeric antigen receptor (CAR) or a transgenic TCR.

In one embodiment, the nucleic acid construct further comprises a nucleic acid sequence encoding a CAR.

In another embodiment, the nucleic acid construct further comprises a nucleic acid sequence encoding a transgenic TCR.

The nucleic acid construct may further comprise a suicide gene. Suitably, the nucleic acid construct may further comprise a nucleic acid sequence encoding a CAR and a suicide gene. Alternatively, the nucleic acid construct may further comprise a nucleic acid sequence encoding a transgenic TCR and a suicide gene.

A suicide-gene is a genetically encoded mechanism which allows selective destruction of adoptively transferred cells, such as T-cells, in the face of unacceptable toxicity. Non-limiting examples of suicide genes include RQR8 (Philip et al. (2014) Blood 124: 1277-87), Herpes Simplex Virus thymidine kinase (HSVtk), inducible Caspase 9 (iCas9), and rapCasp9. The suicide gene preferably allows cells expressing the suicide gene to be selectively deleted in response to administration of a substance. For example, RQR8 facilitates selective deletion of cells expressing this gene upon exposure to rituximab. Similarly, iCasp9 facilitates selective deletion of cells expressing this gene upon exposure to APi 903. Thymidine kinase allows cells expressing this gene to be killed using ganciclovir.

The suicide gene may be “RQR8” as described in WO2013/153391 having the sequence shown as SEQ ID No. 4 of that document; or “Rapcasp9” as described in WO2016/135470.

In embodiments where more than one polypeptide is encoded by a nucleic acid construct, the polypeptides may be encoded as separate reading frames under the control of separate regulatory elements (e.g. separate copies of the same promoter or different promoters). Alternatively or additionally, the polypeptides may be encoded as a single-reading frame under the control of the same regulatory elements (e.g. the same promoter).

Suitably, a co-expression site may be used to enable co-expression of the polypeptides as a single open-reading frame. Typically, a co-expression site is located between the nucleic acid sequences which encode proteins, e.g. the polynucleotide of the invention and the nucleic acid encoding a CAR or transgenic TCR. The co-expression site may comprise a co-expression sequence. The co-expression site may consist of a co-expression sequence.

A co-expression site is used herein to refer to a nucleic acid sequence enabling co-expression of both (i) a CAR or a transgenic TCR; (ii) a polynucleotide of the invention; and optionally (iii) a suicide gene.

The co-expression sequence may be an internal ribosome entry sequence (IRES). The co-expression sequence may be an internal promoter.

The co-expression sequence may encode a cleavage site, such that the nucleic acid construct encodes the polypeptides (e.g. the polypeptide of the invention and nucleic acid encoding a CAR or transgenic TCR) joined by a cleavage site(s).

Suitably, in embodiments where a plurality of co-expression sites are present in the nucleic acid construct, the same co-expression site is used (i.e. the same co-expression site is present between each adjacent pair of nucleic acid sequences which encode proteins).

Preferably, the co-expression site is a cleavage site. The cleavage site may be any sequence which enables the two (or more) polypeptides to become separated. The cleavage site may be self-cleaving, such that when the polypeptide is produced, it is immediately cleaved into individual peptides without the need for any external cleavage activity.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al. (2001) J. Gen. Virol. 82:1027-1041). The exact mechanism of such “cleavage” is not important for the purposes of the present invention, as long as the cleavage site, when positioned between nucleic acid sequences which encode proteins, causes the proteins to be expressed as separate entities.

The cleavage site may be a furin cleavage site.

Furin is an enzyme which belongs to the subtilisin-like proprotein convertase family. The members of this family are proprotein convertases that process latent precursor proteins into their biologically active products. Furin is a calcium-dependent serine endoprotease that can efficiently cleave precursor proteins at their paired basic amino acid processing sites. Examples of furin substrates include proparathyroid hormone, transforming growth factor beta 1 precursor, proalbumin, pro-beta-secretase, membrane type-1 matrix metalloproteinase, beta subunit of pro-nerve growth factor and von Willebrand factor. Furin cleaves proteins just downstream of a basic amino acid target sequence (canonically, Arg-X-(Arg/Lys)-Arg′) and is enriched in the Golgi apparatus.

The cleavage site may be a Tobacco Etch Virus (TEV) cleavage site.

TEV protease is a highly sequence-specific cysteine protease which is chymotrypsin-like proteases. It is very specific for its target cleavage site and is therefore frequently used for the controlled cleavage of fusion proteins both in vitro and in vivo. The consensus TEV cleavage site is ENLYFQ\S (where ‘\’ denotes the cleaved peptide bond). Mammalian cells, such as human cells, do not express TEV protease. Thus in embodiments in which the present nucleic acid construct comprises a TEV cleavage site and is expressed in a mammalian cell—exogenous TEV protease must also expressed in the mammalian cell.

The cleavage site may encode a self-cleaving peptide.

A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donnelly et al. (2001) as above).

“2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al. (2001) as above).

Suitably, the nucleic acid construct may be an operon. An operon is a functioning polynucleotide unit which comprises a plurality of genes under the control of a single promoter. The genes are transcribed together into an mRNA strand and either translated together in the cytoplasm, or undergo trans-splicing to create monocistronic mRNAs that are translated separately, i.e. several strands of mRNA that each encode a single gene product. The result of this is that the genes contained in the operon are either expressed together or not at all.

### Vector

The present invention further provides a vector comprising one or more polynucleotide(s) or nucleic acid construct(s) of the invention.

In some embodiments, the vector further comprises a nucleic acid sequence encoding a CAR or transgenic TCR.

In some embodiments, the vector further comprises a suicide gene.

Such a vector may be used to introduce the polynucleotide(s) or construct(s) into a host cell so that it expresses a polypeptide of the invention. Such a vector may be used to introduce the polynucleotide(s) or construct(s) into a host cell so that it expresses a polypeptide of the invention and a CAR, transgenic TCR and/or a suicide gene.

The vector may comprise one or more polynucleotide(s) or nucleic acid construct(s) of the invention operably linked to a heterologous sequence, such as a promoter or regulatory sequence.

The vector may, for example, be a plasmid or a viral vector, such as a retroviral vector or a lentiviral vector, or a transposon based vector or synthetic mRNA.

The vector may be capable of transfecting or transducing a cell.

### Chimeric Antigen Receptor (CAR)

Classical CARs are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site or on a ligand for the target antigen. A spacer domain may be necessary to isolate the binder from the membrane and to allow it a suitable orientation. A common spacer domain used is the Fc of IgG1. More compact spacers can suffice e.g. the stalk from CD8α and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

CAR-encoding nucleic acids may be transferred to cells (e.g. T-cells or NK cells) using, for example, retroviral vectors. In this way, a large number of antigen-specific cells can be generated for adoptive cell transfer. When the CAR binds the target-antigen, this results in the transmission of an activating signal to the T-cell or NK cell it is expressed on. Thus the CAR directs the specificity and cytotoxicity of the T-cell or NK cell towards cells expressing the targeted antigen.

### Antigen Binding Domain

The antigen-binding domain is the portion of a classical CAR which recognizes antigen.

Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain binder such as a camelid; an artificial binder single as a Darpin; or a single-chain derived from a T-cell receptor. The antibody may be a full-length antibody, a single chain antibody fragment, a F(ab) fragment, a F(ab′)2 fragment, a F(ab′) fragment, a single domain antibody (sdAb), a VHH/nanobody, a nanobody, an affibody, a fibronectin artificial antibody scaffold, an anticalin, an affilin, a DARPin, a VNAR, an iBody, an affimer, a fynomer, a domain antibody (DAb), an abdurin/nanoantibody, a centyrin, an alphabody, a nanofitin or a D domain.

The antibody or antigen binding domain may be non-human, chimeric, humanised or fully human.

Various tumour associated antigens (TAA) are known, as shown in the following Table 1. The antigen-binding domain used in the present invention may be a domain which is capable of binding a TAA as indicated therein.

TABLE 1

| Cancer type | TAA |
|---|---|
| Diffuse Large B-cell Lymphoma | CD19, CD20 |
| Breast cancer | ErbB2, MUC1 |
| AML | CD13, CD33 |
| Neuroblastoma | GD2, NCAM, ALK, GD2 |
| B-CLL | CD19, CD52, CD160 |
| Colorectal cancer | Folate binding protein, CA-125 |
| Chronic Lymphocytic Leukaemia | CD5, CD19 |
| Glioma | EGFR, Vimentin |
| Multiple myeloma | BCMA, CD138 |
| Renal Cell Carcinoma | Carbonic anhydrase IX, G250 |
| Prostate cancer | PSMA |
| Bowel cancer | A33 |

### Transmembrane Domain

The transmembrane domain is the sequence of a classical CAR that spans the membrane. It may comprise a hydrophobic alpha helix. The transmembrane domain may be derived from CD28, which gives good receptor stability.

### Signal Peptide

The CAR may comprise a signal peptide so that when it is expressed in a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

### Spacer Domain

The CAR may comprise a spacer sequence to connect the antigen-binding domain with the transmembrane domain. A flexible spacer allows the antigen-binding domain to orient in different directions to facilitate binding.

The spacer sequence may, for example, comprise an IgG1 Fc region, an IgG1 hinge or a human CD8 stalk or the mouse CD8 stalk. The spacer may alternatively comprise an alternative linker sequence which has similar length and/or domain spacing properties as an IgG1 Fc region, an IgG1 hinge or a CD8 stalk. A human IgG1 spacer may be altered to remove Fc binding motifs.

### Intracellular Signalling Domain

The intracellular signalling domain is the signal-transmission portion of a classical CAR.

The most commonly used signalling domain component is that of CD3-zeta endodomain, which contains 3 ITAMs. This transmits an activation signal to the T-cell after antigen is bound. CD3-zeta may not provide a fully competent activation signal and additional co-stimulatory signalling may be needed. For example, chimeric CD28 and OX40 can be used with CD3-Zeta to transmit a proliferative/survival signal, or all three can be used together.

The intracellular signalling domain may be or comprise a T-cell signalling domain.

The intracellular signalling domain may comprise one or more immunoreceptor tyrosine-based activation motifs (ITAMs). An ITAM is a conserved sequence of four amino adds that is repeated twice in the cytoplasmic tans of certain cell surface proteins of the immune system. The motif contains a tyrosine separated from a leucine or isoleucine by any two other amino acids, giving the signature YxxL/I. Two of these signatures are typically separated by 6 to 8 amino adds in the tail of the molecule (YxxL/Ix₍₆₋₈₎YxxL/I).

ITAMs are important for signal transduction in immune cells. Hence, they are found in the tans of important cell signalling molecules such as the CD3 and ζ-chains of the T-cell receptor complex, the CD79 alpha and beta chains of the B cell receptor complex, and certain Fc receptors. The tyrosine residues within these motifs become phosphorylated following interaction of the receptor molecules with theft ligands and form docking sites for other proteins involved in the signalling pathways of the cell.

The intracellular signalling domain component may comprise, consist essentially of, or consist of the CD3-ζ endodomain, which contains three ITAMs. Classically, the CD3-ζendodomain transmits an activation signal to the T-cell after antigen is bound. However, in the context of the present invention, the CD3-ζ endodomain transmits an activation signal to the T-cell after the MHC/peptide complex comprising the engineered B2M binds to a TCR on a different T-cell.

The intracellular signalling domain may comprise additional co-stimulatory signalling. For example, 4-1BB (also known as CD137) can be used with CD3-ζ, or CD28 and OX40 can be used with CD3-ζ to transmit a proliferative/survival signal.

Accordingly, intracellular signalling domain may comprise the CD3-ζ endodomain alone, the CD3-ζ endodomain in combination with one or more co-stimulatory domains selected from 4-1BB, CD28 or OX40 endodomain, and/or a combination of some or all of 4-1BB, CD28 or OX40.

The endodomain may comprise one or more of the following: an ICOS endodomain, a CD2 endodomain, a CD27 endodomain, or a CD40 endodomain.

The endomain may comprise the sequence shown as SEQ ID NO: 15 to 18 or a variant thereof having at least 80, 85, 90, 95, 98 or 99% sequence identity, provided that the variant sequence retains the capacity to transmit an activating signal to the cell.

The percentage identity between two polypeptide sequences may be readily determined by programs such as BLAST, which is freely available at http://blast.ncbi.nlm.nih.gov. Suitably, the percentage identity is determined across the entirety of the reference and/or the query sequence.

CD3-ζ endodomain
SEQ ID NO: 36
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR
4-1BB and CD3-ζ endodomains
SEQ ID NO: 37
MGNSCYNIVATLLLVLNFERTRSLQDPCSNCPAGTFCDNNRNQICSPCPP
NSFSSAGGQRTCDICRQCKGVFRTRKECSSTSNAECDCTPGFHCLGAGCS
MCEQDCKQGQELTKKGCKDCCFGTFNDQKRGICRPWTNCSLDGKSVLVNG
TKERDVVCGPSPADLSPGASSVTPPAPAREPGHSPQIISFFLALTSTALL
FLLFFLTLRFSVVKRGRKKLLYIFKQPFMRPVQTTQEEDGCSCRFPEEEE
GGCELRVKFSRSADAPAYQQGQNQLYNELNLGRREEYDVLDKRRGRDPEM
GGKPQRRKNPQEGLYNELQKDKMAEAYSEIGMKGERRRGKGHDGLYQGLS
TATKDTYDALHMQALPPR
CD28 and CD3-ζ endodomains
SEQ ID NO: 38
SKRSRLLHSDYMNMTPRRPGPTRKHYQPYAPPRDFAAYRSRVKFSRSADA
PAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALP
PR
CD28, OX40 and CD3-ζ endodomains
SEQ ID NO: 39
SKRSRLLHSDYMNMTPRRPGPTRKHYQPYAPPRDFAAYRSRDQRLPPDAH
KPPGGGSFRTPIQEEQADAHSTLAKIRVKFSRSADAPAYQQGQNQLYNEL
NLGRREEYDVLDKRRGRDPEMGGKPRRKNPQEGLYNELQKDKMAEAYSEI
GMKGERRRGKGHDGLYQGLSTATKDTYDALHMQALPPR

### Transgenic T-Cell Receptor (TCR)

The TCR is a molecule found on the surface of T-cells which is responsible for recognizing fragments of antigen as peptides bound to major histocompatibility complex (MHC) molecules.

The TCR is a heterodimer composed of two different protein chains. In humans, in 95% of T-cells the TCR consists of an alpha (α) chain and a beta (β) chain (encoded by TRA and TRB, respectively), whereas in 5% of T-cells the TCR consists of gamma and delta (γ/δ) chains (encoded by TRG and TRD, respectively).

When the TCR engages with antigenic peptide and MHC (peptide/MHC) complex, the T lymphocyte is activated through signal transduction.

In contrast to conventional antibody-directed target antigens, antigens recognized by the TCR can include the entire array of potential intracellular proteins, which are processed and delivered to the cell surface as a peptide/MHC complex.

It is possible to engineer cells to express heterologous (i.e. non-native) TCR molecules by artificially introducing the TRA and TRB genes; or TRG and TRD genes into the cell using a vector. For example the genes for engineered TCRs may be reintroduced into T-cells isolated form a healthy donor and transferred into patients for T-cell adoptive therapies. Such ‘heterologous’ TCRs may also be referred to herein as ‘transgenic TCRs’.

### Engineered Cell

The present invention provides an engineered cell comprising the polypeptide of the invention, the polynucleotide of the invention, the nucleic acid construct of the invention or the vector of the invention. Preferably, the engineered cell is an allogeneic engineered cell.

In one embodiment, the engineered cell is an engineered T cell or an engineered NK cell.

The engineered cell may further comprise a nucleic acid sequence encoding a CAR or a transgenic TCR. Suitably, the engineered cell may further comprise a nucleic acid sequence encoding a CAR. Alternatively, the engineered cell may further comprise a nucleic acid sequence encoding a transgenic TCR.

The engineered cell may further comprise a suicide gene. Thus, the engineered cell may further comprise a suicide gene and a nucleic acid sequence encoding a CAR or a transgenic TCR.

The engineered cell may have decreased cell surface expression of MHC class I and decreased expression of a TCR or MHC class II compared to a corresponding cell which does not comprise the polypeptide of the invention, the polynucleotide of the invention, the nucleic acid construct of the invention or the vector of the invention.

In one embodiment, the engineered cell has cell surface expression of MHC class I and MHC class II or TCR which is decreased by at least 10% (suitably at least 20%, at least 30%, at least 40%, at least 50%, at least 60%, at least 70%, at least 80% or at least 90%) compared to a corresponding cell which does not comprise the polypeptide of the invention, the polynucleotide of the invention, the nucleic acid construct of the invention or the vector of the invention.

Suitably, MHC class I cell surface expression may be decreased by at least 10% (suitably at least 20%, at least 30%, at least 40%, at least 50%, at least 60%, at least 70%, at least 80% or at least 90%) compared to a corresponding cell which does not comprise the polypeptide of the invention, the polynucleotide of the invention, the nucleic acid construct of the invention or the vector of the invention. Suitably, MHC class II cell surface expression may be decreased by at least 10% (suitably at least 20%, at least 30%, at least 40%, at least 50%, at least 60%, at least 70%, at least 80% or at least 90%) compared to a corresponding cell which does not comprise the polypeptide of the invention, the polynucleotide of the invention, the nucleic acid construct of the invention or the vector of the invention. Suitably, TCR cell surface expression may be decreased by at least 10% (suitably at least 20%, at least 30%, at least 40%, at least 50%, at least 60%, at least 70%, at least 80% or at least 90%) compared to a corresponding cell which does not comprise the polypeptide of the invention, the polynucleotide of the invention, the nucleic acid construct of the invention or the vector of the invention.

The engineered cell of the present invention may be an immune effector cell, such as a T-cell, a natural killer (NK) cell or a cytokine induced killer cell.

The engineered cell may be a cytolytic immune cell such as a T-cell or an NK cell.

The engineered cell may be a natural killer cell (or NK cell). NK cells form part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The engineered cell may be a T-cell and may be an alpha-beta T-cell or a gamma-delta T-cell.

T-cells or T lymphocytes are a type of lymphocyte that play a central role in cell-mediated immunity. They can be distinguished from other lymphocytes, such as B cells and natural killer cells (NK cells), by the presence of a TCR on the cell surface. There are various types of T-cell, as summarised below.

Helper T helper cells (TH cells) assist other white blood cells in immunologic processes, including maturation of B cells into plasma cells and memory B cells, and activation of cytotoxic T-cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, TH9, or TFH, which secrete different cytokines to facilitate different types of immune responses.

Cytolytic T-cells (TC cells, or CTLs) destroy virally infected cells and tumour cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T-cells, the CD8+ cells can be inactivated to an anergic state, which prevent autoimmune diseases such as experimental autoimmune encephalomyelitis.

Memory T-cells are a subset of antigen-specific T-cells that persist long-term after an infection has resolved. They quickly expand to large numbers of effector T-cells upon re-exposure to their cognate antigen, thus providing the immune system with “memory” against past infections. Memory T-cells comprise three subtypes: central memory T-cells (TCM cells) and two types of effector memory T-cells (TEM cells and TEMRA cells). Memory cells may be either CD4+ or CD8+. Memory T-cells typically express the cell surface protein CD45RO.

Regulatory T-cells (Treg cells), formerly known as suppressor T-cells, are crucial for the maintenance of immunological tolerance. Their major role is to shut down T-cell-mediated immunity toward the end of an immune reaction and to suppress auto-reactive T-cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T-cells with both myeloid (CD11c+) and plasmacytoid (CD123+) dendritic cells that have been activated with TSLP.

Naturally occurring Treg cells can be distinguished from other T-cells by the presence of an intracellular molecule called FoxP3. Mutations of the FOXP3 gene can prevent regulatory T-cell development, causing the fatal autoimmune disease IPEX.

Adaptive Treg cells (also known as T0 cells or Th3 cells) may originate during a normal immune response.

Natural killer T-cells (NKT-cells) are a subset of CD1d-restricted T-cells at the interface between the innate and adaptive immune system. NKT-cells recognize lipids and glycolipids presented by CD1d molecules, a member of the CD1 family of antigen-presenting molecules, rather than peptide/MHC complexes. Naturally occurring NKT-cells co-express an αβ TCR and also a variety of molecular markers that are typically associated with NK cells, such as NK1.1, CD16 and CD56 expression and granzyme production. Thus, these cells feature characteristics of both conventional T-cells and NK cells and include both NK1.1+ and NK1.1−, as well as CD4+, CD4−, CD8+ and CD8− cells.

NKT-cells can be subdivided into functional subsets that respond rapidly to a wide variety of glycolipids and stress-related proteins using T- or natural killer (NK) cell-like effector mechanisms. Because of their major modulating effects on immune responses via secretion of cytokines, NKT-cells are also considered important players in tumor immunosurveillance.

The cells of the invention may be any of the cell types mentioned above.

The cell may be derived from a patient's own peripheral blood (1st party), or from HLA-matched or partially HLA-matched donor peripheral blood (2nd party), or unmatched peripheral blood from an unconnected donor (3rd party). Preferably, the cell may be derived from HLA-matched or partially HLA-matched donor peripheral blood (2nd party) or unmatched peripheral blood from an unconnected donor (3rd party). Thus, preferably, the cell is an allogeneic cell.

T or NK cells, for example, may be activated and/or expanded, for example by treatment with an anti-CD3 monoclonal antibody, prior to being transduced with a nucleic acid(s) encoding the polypeptide of the invention, a nucleic acid construct of the invention or a vector of the invention.

Alternatively, the cell may be derived from ex vivo differentiation of inducible progenitor cells or embryonic progenitor cells to T-cells. Alternatively, an immortalized T-cell line which retains its lytic function may be used.

The cell may be a haematopoietic stem cell (HSC). HSCs can be obtained for transplant from the bone marrow of a suitably matched donor, by leukapheresis of peripheral blood after mobilization by administration of pharmacological doses of cytokines such as G-CSF (peripheral blood stem cells (PBSCs)), or from the umbilical cord blood (UCB) collected from the placenta after delivery. The marrow, PBSCs, or UCB may be transplanted without processing, or the HSCs may be enriched by immune selection with a monoclonal antibody to the CD34 surface antigen.

The cell of the present invention is an engineered cell. Accordingly, the nucleic acid sequence encoding a CAR or transgenic TCR and the polynucleotide, nucleic acid construct or vector as described herein which encodes the polypeptide of the invention are not naturally expressed by the cell, e.g. the alpha-beta T-cell, NK cell, gamma-delta T-cell or cytokine-induced killer cell.

### Method of Making an Engineered Cell

Engineered cells (e.g. CAR or transgenic TCR-expressing cells) of the present invention may be generated by introducing DNA or RNA coding for the CAR or TCR or suicide gene and the polypeptide of the invention by one of many means including transduction with a viral vector, transfection with DNA or RNA.

The engineered cell of the invention may be made by introducing:
a) the polynucleotide of the invention, the nucleic acid construct of the invention or the vector of the invention and a nucleic acid sequence encoding a CAR or a transgenic TCR; or
b) the nucleic acid construct or the vector of the invention;
   into a cell.

The cell may be from a sample isolated from a subject or a donor. The sample may be a cell-containing sample.

The cells may be transduced or transfected with one or more of the polynucleotide, the nucleic acid construct or the vector of the invention and a nucleic acid sequence encoding a CAR or a transgenic TCR as defined above in vitro or ex vivo.

The cells may then by purified, for example, selected on the basis of expression of the polypeptide of the invention or the antigen-binding domain of the antigen-binding polypeptide.

As described above, provided is a nucleic acid construct comprising a polynucleotide encoding the polypeptide of the invention wherein the second domain is capable of binding to a component of the TCR/CD3 complex coupled with a nucleic acid sequence encoding a CAR or a transgenic TCR and/or a suicide gene. The nucleic acid construct and vector described herein provides several advantages for the production of engineered cells, for example allogeneic engineered cells for use in adoptive immunotherapy. It will be appreciated by the skilled person that such coupling means that (i) depleting TCR positive cells results in selection of CAR positive, transgenic TCR positive or suicide gene positive cells, and (ii) sorting of CAR positive, transgenic TCR positive or suicide gene positive cells results in selection of TCR negative cells. Therefore, only one sorting step is required to obtain engineered cells suitable for use in an off-the-shelf product.

### Pharmaceutical Composition

The present invention further provides a pharmaceutical composition comprising the polypeptide of the invention, the polynucleotide of the invention, the nucleic acid construct of the invention, the vector of the invention or the engineered cell of the invention.

In one embodiment, the pharmaceutical composition comprises the engineered cell of the invention.

In one embodiment, the pharmaceutical composition comprises a plurality of engineered cells of the invention.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

### Method of Treating and/or Preventing a Disease

The present invention provides a method for treating and/or preventing a disease which comprises the step of administering the engineered cell of the invention, a plurality of engineered cells of the invention or the pharmaceutical composition of the invention to a recipient.

The present invention further provides a method for treating a disease which comprises the step of administering the engineered cell of the invention, a plurality of engineered cells of the invention or the pharmaceutical composition of the invention to a recipient.

The present invention further provides a method for preventing a disease which comprises the step of administering the engineered cell of the invention, a plurality of engineered cells of the invention or the pharmaceutical composition of the invention to a recipient.

In one embodiment, the method comprises the step of administering the engineered cells of the invention to a recipient.

In one embodiment, the method comprises the step of administering the pharmaceutical composition of the invention to a recipient.

In one embodiment, the method comprises the step of administering a plurality of engineered cells of the invention to a recipient.

The present invention further provides the polypeptide of the invention, the polynucleotide of the invention, the nucleic acid construct of the invention, the vector of the invention, the engineered cell of the invention, a plurality of engineered cells of the invention or the pharmaceutical composition of the present invention for use in treating and/or preventing a disease.

The present invention further provides the engineered cell or the pharmaceutical composition of the present invention for use in treating and/or preventing a disease.

In one embodiment, the present invention provides the engineered cell of the invention for use in treating and/or preventing a disease.

In one embodiment, the present invention provides a plurality of engineered cells of the invention for use in treating and/or preventing a disease.

In one embodiment, the present invention provides the pharmaceutical composition of the invention for use in treating and/or preventing a disease.

The invention also relates to the use of the engineered cell or the pharmaceutical composition of the present invention in the manufacture of a medicament for the treatment and/or prevention of a disease.

In one embodiment, the invention relates to the use of the engineered cell of the invention in the manufacture of a medicament for the treatment and/or prevention of a disease.

In one embodiment, the invention relates to the use of the pharmaceutical composition of the invention in the manufacture of a medicament for the treatment and/or prevention of a disease.

A method for treating a disease relates to the therapeutic use of the engineered cells or pharmaceutical composition of the present invention. In this respect, the engineered cells or pharmaceutical composition may be administered to a subject having an existing disease or condition in order to lessen, reduce or improve at least one symptom associated with the disease and/or to slow down, reduce or block the progression of the disease.

A method for preventing a disease relates to the prophylactic use of the engineered cells or pharmaceutical composition of the present invention. In this respect, the engineered cells or pharmaceutical composition may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:
(i) isolation of a cell-containing sample from a subject;
(ii) transduction or transfection of the cells with a polynucleotide, nucleic acid construct or vector of the invention, and optionally with a nucleic acid sequence encoding a CAR or a transgenic TCR; and
(iii) administering the cells obtained in (ii) to a subject.

A “subject” refers to either a human or non-human animal.

Examples of non-human animals include vertebrates, for example mammals, such as non-human primates (particularly higher primates).

Preferably, the subject is a human. The subject may be any age, gender or ethnicity.

In some embodiments, the subject of step (i) and the subject of step (iii) are the same individual.

In some preferred embodiments, the subject of step (i) and the subject of step (iii) are different individuals, i.e. the subject of step (i) is a donor and the subject of step (iii) is a recipient.

Suitably, the donor is fully or partially HLA-matched with the recipient or is not HLA-matched (i.e. is unmatched) with the recipient. Preferably, the donor is not HLA-matched with the recipient.

The subject (recipient) may have a disease, or be thought to be at risk from contracting or developing a disease, because of, for example, family history of the disease or the presence of genetic or phenotypic (e.g. biomarkers) associated with the disease.

The subject (recipient) may show one or more signs or symptoms of a disease. The subject (recipient) may have been previously characterised as having a disease by other diagnostic methods.

The disease to be treated and/or prevented by the method or uses of the present invention may be immune rejection of the engineered cell which comprises (i) a chimeric antigen receptor (CAR) or a transgenic TCR; and (ii) a polypeptide of the invention.

The disease to be treated and/or prevented by the method or uses of the present invention may be a cancerous disease, such as bladder cancer, breast cancer, colon cancer, endometrial cancer, kidney cancer (e.g. renal cell), leukaemia, lung cancer, melanoma, non-Hodgkin lymphoma, pancreatic cancer, prostate cancer and thyroid cancer.

The methods and uses of the invention may be for the treatment of a solid tumour, such as bladder cancer, breast cancer, colon cancer, endometrial cancer, kidney cancer (renal cell), lung cancer, melanoma, neuroblastoma, sarcoma, glioma, pancreatic cancer, prostate cancer and thyroid cancer.

The disease may be a non-solid tumour. For example the disease may be Multiple Myeloma (MM), B-cell Acute Lymphoblastic Leukaemia (B-ALL), Chronic Lymphocytic Leukaemia (CLL), Neuroblastoma, T-cell acute Lymphoblastic Leukaema (T-ALL) or diffuse large B-cell lymphoma (DLBCL).

the disease may be B-cell Acute Lymphoblastic Leukaemia (B-ALL), Chronic Lymphocytic Leukaemia (CLL), T-cell acute Lymphoblastic Leukaema (T-ALL) or diffuse large B-cell lymphoma (DLBCL).

The engineered cell, in particular the CAR cell, or pharmaceutical composition of the present invention may be capable of killing target cells, such as cancer cells. The target cell may be recognisable by expression of a TAA, for example the expression of a TAA provided above in Table 1.

### Method of Reducing and/or Preventing GvHD and/or HvGR

The present invention provides a method of reducing or preventing graft versus host disease (GvHD) and/or host versus graft disease (HvGD) in a subject associated with the administration of one or more engineered cells to the subject, comprising the steps of:
(i) making the one or more engineered cells using the method as described herein; and
(ii) administering the one or more engineered cells to the subject.

The present invention further provides a method of reducing graft versus host disease (GvHD) and/or host versus graft disease (HvGD) in a subject associated with the administration of one or more engineered cells to the subject, comprising the steps of:
(i) making the one or more engineered cells using the method as described herein; and
(ii) administering the one or more engineered cells to the subject.

The present invention further provides a method of preventing graft versus host disease (GvHD) and/or host versus graft disease (HvGD) in a subject associated with the administration of one or more engineered cells to the subject, comprising the steps of:
(i) making the one or more engineered cells using the method as described herein; and
(ii) administering the one or more engineered cells to the subject.

In one embodiment, the method reduces or prevents GvHD.

In one embodiment, the method reduces or prevents HvGD.

In one embodiment, the method reduces or prevents GvHD and HvGD.

In one embodiment, the method comprises the steps of:
(i) introducing:
   a) the polynucleotide of the invention, the nucleic acid construct of the invention or the vector of the invention and a nucleic acid sequence encoding a CAR or a transgenic TCR; or
   b) the nucleic acid construct or the vector of the invention; into one or more cells; and
(ii) administering the one or more cells to the subject.

The method may involve the steps of:
(i) isolating a cell-containing sample from a subject;
(ii) transducing or transfecting of the cells with a polynucleotide, nucleic acid construct or vector of the invention, and optionally with a nucleic acid sequence encoding a CAR or a transgenic TCR; and
(iii) administering the cells obtained in (ii) to a subject.

The subject may be a subject as described herein.

In one embodiment, the administration of the one or more cells to the subject is by transfusion. Preferably, the one or more cells may be an allogeneic engineered cell.

Thus, the present invention provides a method of reducing or preventing GvHD and/or HvGD during adoptive immunotherapy with engineered cells, preferably with allogeneic engineered cells.

### Kit

The present invention provides a kit comprising the polynucleotide of the invention and a nucleic acid sequence encoding a CAR or a transgenic TCR, the nucleic acid construct of the invention or the vector of the invention.

The present invention further provides a kit comprising:
(i) a vector comprising the polynucleotide of the invention and a vector comprising a nucleic acid sequence encoding a CAR, or a transgenic TCR, optionally further comprising a suicide gene; or
(ii) a vector comprising the polynucleotide of the invention and a nucleic acid sequence encoding a CAR or a transgenic TCR, optionally further including a suicide gene.

### Dosage

The skilled person can readily determine an appropriate dose of one of the agents of the invention to administer to a subject without undue experimentation. Typically, a physician will determine the actual dosage which will be most suitable for an individual patient and it will depend on a variety of factors including the activity of the specific agent employed, the metabolic stability and length of action of that agent, the age, body weight, general health, sex, diet, mode and time of administration, rate of excretion, drug combination, the severity of the particular condition, and the individual undergoing therapy. There can of course be individual instances where higher or lower dosage ranges are merited, and such are within the scope of the invention.

The skilled person appreciates, for example, that route of delivery (e.g. oral vs. intravenous vs. subcutaneous etc.) may impact the required dosage (and vice versa). For example, where particularly high concentrations of an agent within a particular site or location are desired, focussed delivery may be preferred. Other factors to be considered when optimizing routes and/or dosing schedule for a given therapeutic regimen may include, for example, the disease being treated (e.g. type or stage etc.), the clinical condition of a subject (e.g. age, overall health etc.), the presence or absence of combination therapy, and other factors known to medical practitioners.

The dosage is such that it is sufficient to improve symptoms or markers of the disease—as described herein.

### Administration

Suitably, the engineered cell or pharmaceutical composition of the invention will be formulated for administration by injection, for example by intramuscular, intradermal, intravenous or sub-cutaneous injection.

An engineered cell or a pharmaceutical composition of the invention will generally be administered in admixture with a pharmaceutical carrier, excipient or diluent, particularly for human therapy.

### Variants, Homologues and Fragments

In addition to the specific proteins and nucleotides mentioned herein, the invention also encompasses the use of variants, derivatives, analogues, homologues and fragments thereof.

In the context of the invention, a variant of any given sequence is a sequence in which the specific sequence of residues (whether amino acid or nucleic acid residues) has been modified in such a manner that the polypeptide or polynucleotide in question substantially retains its function. A variant sequence can be obtained by addition, deletion, substitution, modification, replacement and/or variation of at least one residue present in the naturally-occurring protein.

The term “derivative” as used herein, in relation to proteins or polypeptides of the invention includes any substitution of, variation of, modification of, replacement of, deletion of and/or addition of one (or more) amino acid residues from or to the sequence providing that the resultant protein or polypeptide substantially retains at least one of its endogenous functions.

The term “analogue” as used herein, in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3 to 10 or 20 substitutions provided that the modified sequence substantially retains the required activity or ability. Amino acid substitutions may include the use of non-naturally occurring analogues.

Proteins used in the invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

The term “homologue” as used herein means an entity having a certain homology with the wild type amino acid sequence and the wild type nucleotide sequence. The term “homology” can be equated with “identity”.

A homologous sequence may include an amino acid sequence which may be at least 50%, 55%, 60%, 65%, 70%, 75%, 80%, 85% or 90% identical, preferably at least 95% or 97% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the invention it is preferred to express homology in terms of sequence identity.

A homologous sequence may include a nucleotide sequence which may be at least 50%, 55%, 60%, 65%, 70%, 75%, 80%, 85% or 90% identical, preferably at least 95% or 97% or 99% identical to the subject sequence. Although homology can also be considered in terms of similarity, in the context of the invention it is preferred to express homology in terms of sequence identity.

Preferably, reference to a sequence which has a percent identity to any one of the SEQ ID NOs detailed herein refers to a sequence which has the stated percent identity over the entire length of the SEQ ID NO referred to.

Homology comparisons can be conducted by eye or, more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174: 247-50; FEMS Microbiol. Lett. (1999) 177: 187-8).

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent homology, preferably percent sequence identity. The software typically does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Example 1

The anti-CD3 scFv derived from the hybridoma UCHT1 was cloned as an N-terminal fusion with US11. The sequence of the UCHT1-US11 fusion protein is shown in FIG. 3.

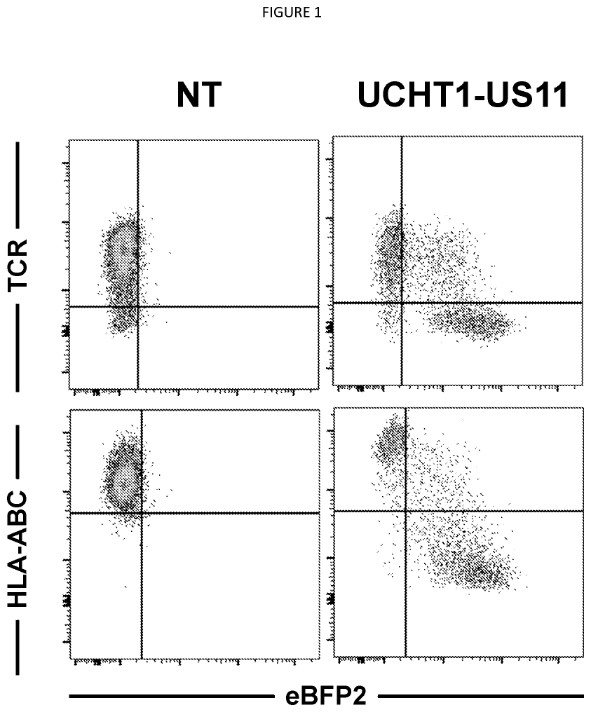

Primary T-cells from a healthy donor were mock transduced or transduced with a retroviral vector expressing UCHT1-US11 fusion protein and blue fluorescent protein (BFP) marker followed by staining for expression of TCR and HLA-ABC. The clone of TCR staining antibody was chosen so as to not compete with UCHT1 binding. We demonstrated that in transduced cells there was downregulation of staining for both TCR and HLA, which was reduced to the level of isotype-control stained cells (FIG. 1). Downregulation of both TCR and HLA-ABC was seen only in transduced cells.

Similar results were also achieved with a UCHT1-US11 fusion protein comprising a Ser-Gly linker between the UCHT1 and US11 domains.

### Example 2

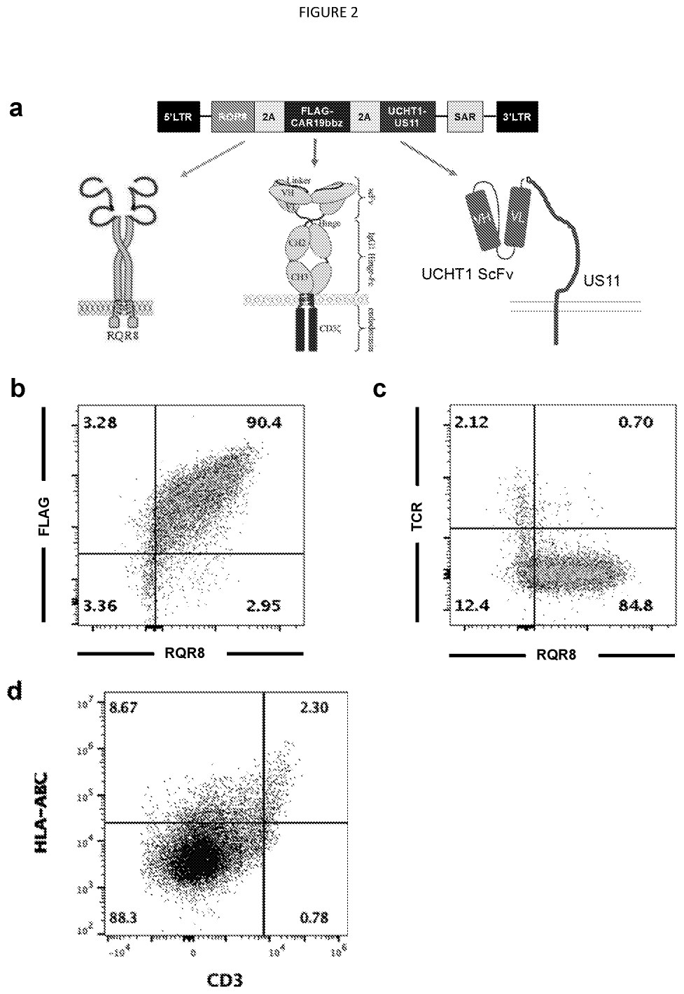

We subsequently generated a tricistronic vector in which we co-expressed a sort-suicide gene (RQR8), a 2^{nd} generation anti-CD19 CAR and UCHT1-US11 separated by 2A peptides (FIG. 2A). We demonstrated that transduced cells demonstrated cell surface expression of RQR8 and CAR (FIG. 2B), with downregulation of cell surface expression of TCR (FIG. 2C) and HLA-ABC (FIG. 2D). Importantly, there was obligate linkage of these parameters. Further, after positive immunomagnetic selection for the RQR8 protein, we obtained >97% pure populations of transduced cells (US11CAR).

### Example 3

The anti-CD3 scFv derived from the hybridoma UCHT1 was cloned as an N-terminal fusion with E19. The sequence of the UCHT1-E19 fusion protein is shown as SEQ ID NO: 40.

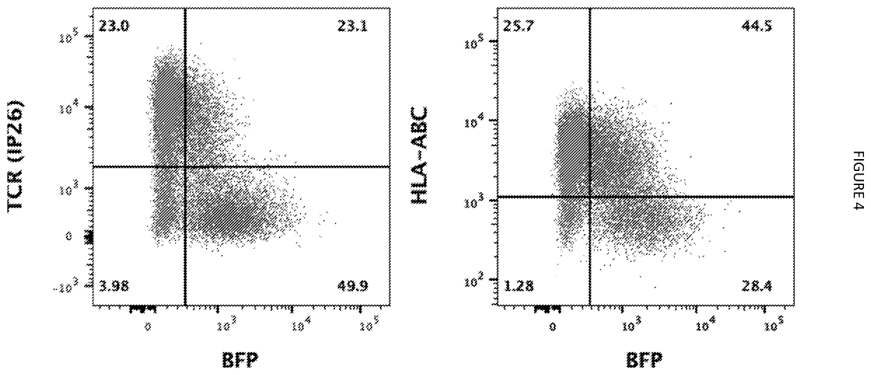

Jurkat cells were transduced with a retroviral vector expressing UCHT1-E19 fusion protein and blue fluorescent protein (BFP) marker followed by staining for expression of TCR and HLA-ABC. In transduced cells there was downregulation of staining for both TCR and HLA (FIG. 4).

SEQ ID NO: 40
DIQMTQSPSSLSASVGNRVTITCRASQDIRNYLNWYQQKPGKAPKLLIYY
TSRLESGVPSRFSGSGSGTDYTLTISSLQPEDFATYYCQQGNTLPWTFGQ
GTKVEIKSGGGGSGGGGSGGGGSEVQLVESGGGLVQPGGSLRLSCAASGY
SFTGYTMNWVRQAPGKGLEWVALINPYKGVSTYNQKFKDRFTISVDKSKN
TAYLQMNSLRAEDTAVYYCARSGYYGDSDWYFDVWGQGTLVTVSSDPTQK
VDFKEPACNVTFAAEANECTTLIKCTTEHEKLLIRHKNKIGKYAVYAIWQ
PGDTTEYNVTVFQGKSHKTFMYTFPFYEMCDITMYMSKQYKLWPPQNCVE
NTGTFCCTAMLITVLALVCTLLYIKYKSRRSFIEEKKMP

### Example 4

An anti-CD3 VHH was cloned as an N-terminal fusion with US11. The sequence of the antiCD3 VHH-US11 fusion protein is shown as SEQ ID NO: 41.

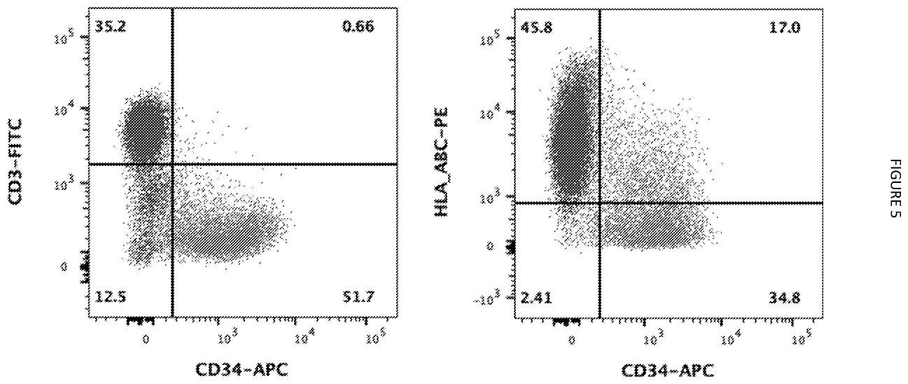

Primary human T cells were mock transduced or transduced with a retroviral vector expressing antiCD3 VHH-US11 fusion protein and CD34 marker gene followed by staining for expression of TCR and HLA-ABC. In transduced cells there was downregulation of staining for both TCR and HLA (FIG. 5).

SEQ ID NO: 41
EVQAESGGGSVQTGGSLRLSCAYTASSVCMAWFRQAPGKEREGVAVTREG
LTKTGYADSVKGRFAISQDYAKKTLYLQMSSLKPEDTARYYCAARPTSPC
TVDGELLASTYNYWGQGTQVTVSSDPMPELSLTLFDEPPPLVETEPLPPL
PDVSEYRVEYSEARCVLRSGGRLEALWTLRGNLSVPTPTPRVYYQTLEGY
ADRVPTPVEDISESLVAKRYWLRDYRVPQRTKLVLFYFSPCHQCQTYYVE
CEPRCLVPWVPLWSSLEDIERLLFEDRRLMAYYALTIKSAQYTLMMVAVI
QVFWGLYVKGWLHRHFPWMFSDQW

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A polypeptide comprising:
(i) a first domain which is capable of downregulating cell surface expression of an MHC class I molecule; and
(ii) a second domain which is capable of binding a target molecule.

2. The polypeptide according to claim 1, wherein the target molecule is a component of T-cell receptor (TCR)/CD3 complex or MHC class II.

3. The polypeptide according to claim 1 or claim 2, wherein the second domain is an antibody or a fragment thereof.

4. The polypeptide according to any one of the preceding claims, wherein the second domain is selected from an scFv, a full-length antibody, a single chain antibody fragment, a F(ab) fragment, a F(ab′)2 fragment, a F(ab′) fragment, a single domain antibody (sdAb), a VHH/nanobody and a nanobody.

5. The polypeptide according to any one of the preceding claims, wherein the second domain is an scFv.

6. The polypeptide according to any one of the preceding claims, wherein the second domain comprises:
(i) a sequence comprising the complementarity determining regions (CDRs) as shown in any one of SEQ ID NO: 19, SEQ ID NO: 21, SEQ ID NO: 23 or SEQ ID NO: 25; or
(ii) a sequence as set forth in SEQ ID NO: 19, SEQ ID NO: 21, SEQ ID NO: 23, SEQ ID NO: 25 or SEQ ID NO: 42, or a sequence having at least 80% identity thereto or a functional fragment thereof.

7. The polypeptide according to any one of the preceding claims, wherein the second domain comprises: (i) a sequence comprising the CDRs as shown in SEQ ID NO: 19; or (ii) a sequence as set forth in SEQ ID NO: 19, or a sequence having at least 80% identity thereto or a functional fragment thereof.

8. The polypeptide according to any one of the preceding claims, wherein the polypeptide further comprises a signal peptide.

9. The polypeptide according to any one of the preceding claims, wherein the first domain is a viral protein which is capable of downregulating cell surface expression of an MHC class I molecule or a functional fragment thereof.

10. The polypeptide according to any one of the preceding claims, wherein the first domain is a viral protein, or a functional fragment thereof, which:
(i) induces MHC class I translocation from the ER to the cytosol and subsequent MHC class I degradation by the proteasome;
(ii) prevents transport of the MHC class I molecule from the ER to the plasma membrane; and/or
(iii) induces internalisation of the MHC class I molecule via endocytosis and subsequent degradation or sequestration of the MHC class I molecule.

11. The polypeptide according to any one of the preceding claims, wherein the first domain, when expressed in a cell, decreases cell surface expression of an MHC class I molecule by at least 10%.

12. The polypeptide according to any one of the preceding claims, wherein the first domain comprises human cytomegalovirus (HCMV) unique short 11 (US11), HCMV US2, adenovirus E3/19K (E19), HCMV US3, HCMV US10, human herpesvirus 7 (HHV-7) U21, human immunodeficiency virus type 1(HIV-1) negative factor (Nef), Kaposi's sarcoma-associated herpesvirus (KSHV) K3, KSHV K5, or a functional fragment thereof.

13. The polypeptide according to any one of the preceding claims, wherein the first domain comprises HCMV US11 or HCMV US2, or a functional fragment thereof.

14. The polypeptide according to any one of the preceding claims, wherein the first domain is HCMV US11 or a functional fragment thereof.

15. The polypeptide according to any one of the preceding claims, wherein the first domain comprises a sequence as set forth in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 43 or a sequence having at least 80% identity thereto or a functional fragment thereof.

16. The polypeptide according to any one of the preceding claims, wherein the first domain comprises a sequence as set forth in SEQ ID NO: 1, SEQ ID NO: 3, SEQ ID NO: 43 or a sequence having at least 80% identity thereto or a functional fragment thereof.

17. The polypeptide according to any one of the preceding claims, wherein (i) the first domain is directly fused to the second domain; or (ii) the first domain is connected to the second domain by a linker sequence.

18. A polynucleotide encoding the polypeptide according to any one of the preceding claims.

19. A nucleic acid construct comprising the polynucleotide of claim 18.

20. The nucleic acid construct according to claim 19, wherein the nucleic acid construct further comprises a nucleic acid sequence encoding a chimeric antigen receptor (CAR) or a transgenic TCR, preferably wherein the nucleic acid construct further comprises a suicide gene.

21. A vector comprising the polynucleotide of claim 18 or the nucleic acid construct of claim 19 or claim 20.

22. An engineered cell comprising the polypeptide of any one of claims 1-17, the polynucleotide of claim 18, the nucleic acid construct of claims 19 or claim 20 or the vector of claim 21.

23. The engineered cell according to claim 22, wherein the engineered cell is an allogeneic cell.

24. The engineered cell according to claim 22 or claim 23, wherein the engineered cell is an engineered T cell or an engineered NK cell.

25. The engineered cell according to any one of claims 22 to 24, wherein the engineered cell further comprises a nucleic acid sequence encoding a CAR, a transgenic TCR and/or a suicide gene.

26. The engineered cell according to any one of claims 22 to 25, wherein the engineered cell has decreased cell surface expression of MHC class I and decreased expression of the target molecule compared to a cell which does not comprise the polypeptide of any one of claims 1-20 17, the polynucleotide of claim 18, the nucleic acid construct of claim 19 or claim 20 or the vector of claim 21.

27. A method for making an engineered cell according to any of claims 22 to 26, which comprises the step of introducing:
(a) the polynucleotide of claim 18, the nucleic acid construct of claim 19 or the vector of claim 21 and a nucleic acid sequence encoding a CAR or a transgenic TCR; or
(b) the nucleic acid construct of claim 20 or the vector of claim 21;
into a cell.

28. The method according to claim 27, wherein the cell is from a sample isolated from a donor.

29. A pharmaceutical composition comprising the polypeptide of any one of claims 1 to 17, the polynucleotide of claim 18, the nucleic acid construct of claim 19 or claim 20, the vector of claim 21 or an engineered cell according to any of claims 22 to 26.

30. A method for treating and/or preventing a disease comprising the step of administering an engineered cell according to any of claims 22 to 26, a population of engineered cells according to any of claims 22 to 26 or the pharmaceutical composition of claim 29 to a recipient.

31. The method according to claim 30, wherein the donor is different to the recipient.

32. The method according to claim 30 or claim 31, wherein the engineered cell is an allogeneic engineered cell or the population of engineered cells is a population of allogeneic engineered cells.

33. The method according to any one of claims 30 to 32, wherein the recipient is not HLA typed.

34. The method according to any one of claims 30 to 33, wherein the donor is not HLA matched to the recipient.

35. The method according to any one of claims 30 to 34, wherein the method comprises the following steps:
(i) isolation of a cell-containing sample from the donor;
(ii) transduction or transfection of the cells following the method according to claim 26; and
(iii) administering the cells obtained in (ii) to the recipient.

36. An engineered cell according to any of claims 22 to 26 or a pharmaceutical composition according to claim 29 for use in treating and/or preventing a disease.

37. The engineered cell for use according to claim 36, wherein the engineered cell is an allogeneic engineered cell or the population of engineered cells is a population of allogeneic engineered cells.

38. Use of an engineered cell according to any of claims 22 to 26 in the manufacture of a medicament for treating and/or preventing a disease.

39. The method according to any one of claims 30 to 35, or the engineered cell for use according to claim 36 or claim 37, or the pharmaceutical composition for use according to claim 36 or claim 37, or the use according to claim 38, wherein the disease is a cancer.

40. A method of reducing or preventing graft versus host disease (GvHD) and host versus graft disease (HvGD) in a subject associated with the administration of one or more engineered cells to the subject, comprising the steps of:
(i) making the one or more engineered cells according to the method of claim 27 or claim 28; and
(ii) administering the one or more engineered cells to the subject.

41. A method of reducing or preventing
GvHD in a subject associated with the administration of one or more engineered cells to the subject, comprising the steps of:
(i) making the one or more engineered cells according to the method of claim 27 or claim 28; and
(ii) administering the one or more engineered cells to the subject.

42. A method of reducing or preventing HvGD in a subject associated with the administration of one or more engineered cells to the subject, comprising the steps of:
making the one or more engineered cells according to the method of claim 27 or claim 28; and
(ii) administering the one or more engineered cells to the subject.

43. The method according to any of claims 40-42, wherein the engineered cell is an allogeneic engineered cell.

44. A kit comprising the polynucleotide of claim 18 and a nucleic acid sequence encoding a CAR and/or a transgenic TCR, the nucleic acid construct of claim 19 or claim 20 or the vector of claim 21.

45. A kit comprising:
(i) a vector comprising the polynucleotide of claim 18 and a vector comprising a nucleic acid sequence encoding a CAR or a transgenic TCR; or
(ii) a vector comprising the polynucleotide of claim 18 and a nucleic acid sequence encoding a CAR or a transgenic TCR.


SEQUENCE LISTING (45 sequences)

SEQ ID NO: 1 - protein, 215 aa, Artificial Sequence
  HCMV US11
MNLVMLILALWAPVAGSMPELSLTLFDEPPPLVETEPLPPLSDVSEYRVEYSEARCVLRSGGRLEALWTLRGNLSVPTPTPRVYYQTLEGYADRVPTPVEDVSESLVAKRYWLRDYRVPQRTKLVLFYFSPCHQCQTYYVECEPRCLVPWVPLWSSLEDIERLLFEDRRLMAYYALTIKSAQYTLMMVAVIQVFWGLYVKGWLHRHFPWMFSDQW

SEQ ID NO: 2 - DNA, 648 bp, Artificial Sequence
  HCMV US11
atgaaccttgtaatgcttattctagccctctgggccccggtcgcgggtagtatgcctgaattatccttgactcttttcgatgaacctccgcccttggtggagacggagccgttaccgcctctgtccgatgtttcggagtaccgagtagagtattccgaggcgcgctgcgtgctccgatcgggcggtcgactggaggctctgtggaccctgcgcgggaacctgtccgtgcccacgccgacaccccgggtgtactaccagacgctggagggctacgcggatcgagtgccgacgccggtggaggacgtctccgaaagcctcgtcgcaaaacgctactggctccgggactatcgtgttccccaacgcacaaaactcgtgttgttctacttttccccctgccaccaatgccaaacttattatgtagagtgcgaaccccggtgcctcgtgccttgggttcccctgtggagctcgttagaggacatcgaacgactattgttcgaagatcgccgtctaatggcgtactacgcgctcacgattaagtcggcgcagtatacgctgatgatggtggcagtgattcaagtgttttgggggctgtatgtgaaaggttggctgcaccgacattttccctggatgttttcggaccagtggtga

SEQ ID NO: 3 - protein, 199 aa, Artificial Sequence
  HCMV US2
MNNLWKAWVGLWTSMGPLIRLPDGITKAGEDALRPWKSTAKHPWFEIEDNRCYIDNGKLFARGSIVGNMSRFVFDPKADYGGVGENLYVHADDVEFVPGESLKWNVRNLDVMPIFETLALRLVLQGDVIWLRCVPELRVDYTSSAYMWNMQYGMVRKSYTHVAWTIVFYSINITLLVLFIVYVTVDCNLSMMWMRFFVC

SEQ ID NO: 4 - DNA, 600 bp, Artificial Sequence
  HCMV US2
atgaacaatctctggaaagcctgggtgggtctttggacctccatgggtcccttgatccgcctgcccgatggcatcactaaagccggggaagacgcgctccggccctggaagtccacggccaagcacccctggtttgagatcgaggacaaccggtgctacattgacaacggcaagttgtttgctcgggggagcatcgtgggcaacatgagtcggttcgtcttcgatccgaaggccgattatggcggcgtgggagagaacctgtacgtacacgccgacgacgtggagttcgttcccggggagtcgttaaagtggaacgtgcggaacttagatgtgatgccgatcttcgagacgctagccctgcgtctggtactgcaaggggatgtgatctggctgcgttgcgtccccgaactgcgagtggattacacgtctagcgcgtacatgtggaacatgcagtacgggatggtgcggaagtcatacacgcatgtggcctggacaatagtgttttactccataaacattaccctgttggtattgtttatcgtgtatgtgactgtggactgtaacttgtctatgatgtggatgcggtttttcgtgtgctaa

SEQ ID NO: 5 - protein, 159 aa, Artificial Sequence
  adenovirus E19
MRYMILGLLALAAVCSAAKKVEFKEPACNVTFKSEANECTTLIKCTTEHEKLIIRHKDKIGKYAVYAIWQPGDTNDYNVTVFQGENRKTFMYKFPFYEMCDITMYMSKQYKLWPPQKCLENTGTFCSTALLITALALVCTLLYLKYKSRRSFIDEKKMP

SEQ ID NO: 6 - DNA, 480 bp, Artificial Sequence
  adenovirus E19
atgaggtacatgattttaggcttgctcgcccttgcggcagtctgcagcgctgccaaaaaggttgagtttaaggaaccagcttgcaatgttacatttaaatcagaagctaatgaatgcactactcttataaaatgcaccacagaacatgaaaagcttattattcgccacaaagacaaaattggcaagtatgctgtatatgctatttggcagccaggtgacactaacgactataatgtcacagtcttccaaggtgaaaatcgtaaaacttttatgtataaatttccattttatgaaatgtgcgatattaccatgtacatgagcaaacagtacaagttgtggcccccacaaaagtgtttagagaacactggcaccttttgttccaccgctctgcttattacagcgcttgctttggtatgtaccttactttatctcaaatacaaaagcagacgcagttttattgatgaaaagaaaatgccttga

SEQ ID NO: 7 - protein, 186 aa, Artificial Sequence
  HCMV US3
MKPVLVLAILAVLFLRLADSVPRPLNVVVSEIKSAHFRVEENQCWFHMGMLYFKGRMSGNFTKKHFVNVGIVSQSYMDRLQVSGEQYHHDERGAYFEWNIGGYPVSHTVDMVDITLSTRWGDPKKYAACVPQVRMDYSSQTINWYLQRSMRDDNWGLLFRTLLVYLFSLVVLVLLTVGVSARLRFI

SEQ ID NO: 8 - DNA, 561 bp, Artificial Sequence
  HCMV US3
atgaagccggtgttggtgctcgcgatcctggccgtcctgttcctgagactcgcggactcggtgccccggcccctgaatgtggtggtatcggagatcaaatcggcccactttcgggtggaggagaaccaatgctggttccatatgggcatgctgtacttcaaggggaggatgtcgggcaacttcaccaagaaacactttgtgaacgtgggtatcgtctctcaatcttacatggacagattgcaagtgtccggtgagcagtaccaccatgatgagcgcggagcctatttcgagtggaacatcggtgggtacccggtgtcgcacaccgtggatatggtggacatcacgttgtctacgaggtggggagaccccaagaaatatgccgcgtgtgttccccaggtgaggatggactatagctctcagaccatcaactggtaccttcagcgaagcatgagagacgacaattggggtctgctgttcagaaccctgctcgtatatctgttctctctggttgttcttgtgctcctgaccgtaggggtgagcgcccgtctgcgatttatttaa

SEQ ID NO: 9 - protein, 185 aa, Artificial Sequence
  HCMV US10
MLRRGSLRNPLAICLLWWLGVVAAATEETREPTYFTCGCVIQNHVLKGAVKLYGQFPSPKTLRASAWLHDGENHERHRQPILVEGTATATEALYILLPTELSSPEGNRPRNYSATLTLASRDCYERFVCPVYDSGTPMGVLMNLTYLWYLGDYGAILKIYFGLFCGACVITRSLLLICGYYPPRE

SEQ ID NO: 10 - DNA, 558 bp, Artificial Sequence
  HCMV US10
atgctacgccggggaagcctccggaaccctctggcgatctgcctgttgtggtggctgggagtggtggcggcagccacggaggagacgagagaacctacctacttcacgtgcggctgtgttattcagaaccatgtactgaagggtgcggtgaaactctatggacaattcccctcgcctaagactttgcgggcctcggcttggctacacgacggggaaaatcacgaaaggcaccggcagcccatcctcgtggaaggcaccgcgacagctaccgaggcgctgtacattctgctgcccacggaactgtcgtcgccggaaggaaaccgaccccgaaactactctgctaccctaacactcgcctcccgggattgttatgaacgcttcgtgtgcccggtatacgattccgggacgccgatgggcgttttgatgaacttaacgtacctctggtatctaggcgactatggggcaatactaaaaatttatttcggactgttctgcggggcctgcgtcatcacccgatccctcctcctgatatgcggttattatccacctcgcgaataa

SEQ ID NO: 11 - protein, 430 aa, Artificial Sequence
  HHV-7 U21
MWTILLFCVPVIYGELYPDFCPLAVVDFDVNATVDDLLLFDISLSKQCSDDKIRHSAVAAMTDNAFFFGNSETQIETDFGKYLAFNCYQVFSTLNHFLFKNFKKTKGLMKRYDKLCLDVESYIHIQIICSPFKSFIRLRRMNETGISPRILETTFYLQNKRNSTWVAIKNYLGEDDPFTYRIWHTLTHAKNFLINSCENDFNQLFFWQRKYLSLAKTFEATFKQGFNPMIEQRNEQRYRTNNIDCSFSKFRQNGVKVAVCKYTGWGVSGFGSLEVLQKIKSPFGEEWKRVGFNSTGAFTPLYGSDVLWGLIFLRVEMTTYVCTCTNKNTGTQIQVTLPDVDLDLLDSEKTSSNVFVDMLCYTLIAILFLAFVTAVVLLGVSCLDGVQKVLTWPLQHIQKEPVSEKIINLTNLMFGQEPLPKKESLKQQCL

SEQ ID NO: 12 - DNA, 1293 bp, Artificial Sequence
  HHV-7 U21
atgtggactatcttgctgttttgcgttcctgtgatttatggagaactctatccggatttctgtcccttagcagttgtggattttgatgtgaatgctacagtggatgatttgctattgttcgacatatctttgagcaaacaatgttcagacgataaaatcagacactctgcagtcgcggcaatgacagacaatgctttcttttttggcaacagcgaaacgcaaattgaaaccgattttggcaaatatttggctttcaactgttatcaagtattttctacactaaatcattttttgtttaagaactttaaaaaaacaaaaggactgatgaagcgatatgataaactctgtcttgatgtcgagtcttacattcatattcaaattatctgttcaccctttaaaagttttatccgtctgagacgtatgaatgaaactggaatttctcctagaattcttgaaaccacattctacttgcaaaataaaagaaacagcacgtgggtagcgattaaaaactatttaggtgaagatgatcccttcacttatcgaatttggcacactttgacacacgctaaaaactttctgatcaattcttgtgaaaatgacttcaatcaactttttttttggcaaagaaagtacctatcactcgcgaaaacattcgaagctacatttaagcaaggctttaatcctatgattgaacaaagaaatgaacaacgatatagaactaataacatagattgttcattttcgaaatttagacagaatggtgtaaaggtagcagtttgtaaatatacaggatggggtgttagcggtttcggctccttagaagttctgcaaaagataaaaagtccgtttggagaagagtggaaaagagttggtttcaactctacgggtgcttttactcctttgtatggtagtgatgtgctttggggtctaatttttctaagagtagaaatgaccacatatgtttgtacctgtacgaataagaacacaggaacacaaatacaggtaacattacctgatgtggatttggatctattagatagtgaaaaaacgtctagtaatgttttcgtagacatgctttgctacacattgattgctatattattccttgcatttgttacagcggtagttttattaggagtttcctgtttagacggtgttcagaaagttctgacgtggcctttacagcacatacaaaaagaaccagtatcagagaaaataatcaatctaacaaacttgatgtttggacaagaaccgttacctaaaaaagaatctctcaaacagcaatgtttgtga

SEQ ID NO: 13 - protein, 206 aa, Artificial Sequence
  HIV-1 Nef
MGGKWSKSSVIGWPTVRERMRRAEPAADRVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKIEEANKGENTSLLHPVSLHGMDDPEREVLEWRFDSRLAFHHVARELHPEYFKNC

SEQ ID NO: 14 - DNA, 621 bp, Artificial Sequence
  HIV-1 Nef
atgggtggcaagtggtcaaaaagtagtgtgattggatggcctactgtaagggaaagaatgagacgagctgagccagcagcagatagggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataagatagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctga

SEQ ID NO: 15 - protein, 322 aa, Artificial Sequence
  KSHV K3
MEDEDVPVCWICNEELGNERFRACGCTGELENVHRSCLSTWLTISRNTACQICGVVYNTRVVWRPLREMTLLPRLTYQEGLELIVFIFIMTLGAAGLAAATWVWLYIVGGHDPEIDHVAAAAYYVFFVFYQLFVVFGLGAFFHMMRHVGRAYAAVNTRVEVFPYRPRPTSPECAVEEIELQEILPRGDNQDEEGPAGAAPGDQDGPADGAPVHRDSEESVDEAAGYKEAGEPTHNDGRDDNVEPTAVGCDCNNLGAERYRATYCGGYVGAQSGDGAYSVSCHNKAGPSSLVDILPQGLLGGGYGSMGVIRKRSAVSSALMFH

SEQ ID NO: 16 - DNA, 970 bp, Artificial Sequence
  KSHV K3
atggaagatgaggatgttcctgtctgctggatttgcaacgaggagctcggaaatgagagatttagagcctgtggatgcacaggagagctcgagaacgtccatagaagttgtttaagcacctggctcactatctctagaaacacggcctgtcagatatgtggcgtcgtatacaacacgcgcgtggtctggcgacccttgcgcgaaatgacgctattgcctcggctgacttaccaggagggtctggaactgattgtttttattttcatcatgacattgggggccgctggccttgccgctgcgacgtgggtttggctatatatagtgggcggtcatgacccagagatagatcacgtcgctgcagccgcgtactacgtttttttcgtgttttaccaattgtttgtcgtttttgggttgggtgcgtttttccacatgatgcgccacgtggggcgggcatacgctgctgtaaacacccgggttgaagtgtttccatatagacctcggccgacatcaccagagtgtgcggtagaggagatcgagcttcaggaaattcttccccgtggggataaccaggacgaggaggggcccgcgggggcagctccaggcgaccaagatggccccgcggatggcgctcctgtgcatcgcgactcagaagaatccgtggatgaagctgcagggtacaaggaagcgggagaaccaacacataatgatggacgtgatgacaatgtagagccaaccgcggttgggtgtgactgtaacaacttgggcgctgagcggtatagggccacttactgtggcggttatgttggtgcccagtcgggcgatggggcttatagtgtctcctgccataataaggctggaccctcctctctagttgatatccttccacagggtttgcttgggggtggctatggttccatgggcgtgattaggaaacgttcggctgtttcgtctgcccttatgtttcattaaa

SEQ ID NO: 17 - protein, 256 aa, Artificial Sequence
  KSHV K5
MASKDVEEGVEGPICWICREEVGNEGIHPCACTGELDVVHPQCLSTWLTVSRNTACQMCRVIYRTRTQWRSRLNLWPEMERQEIFELFLLMSVVVAGLVGVALCTWTLLVILTAPAGTFSPGAVLGFLCFFGFYQIFIVFAFGGICRVSGTVRALYAANNTRVTVLPYRRPRRPTANEDNIELTVLVGPAGGTDEEPTDESSEGDVASGDKERDGSSGDEPDGGPNDRAGLRGTARTDLCAPTKKPVRKNHPKNNG

SEQ ID NO: 18 - DNA, 771 bp, Artificial Sequence
  KSHV K5
atggcgtccaaggacgtagaagagggtgtagagggacccatctgctggatatgtagggaagaggtggggaacgagggcatacacccctgcgcctgtaccggagagctggatgtcgtccacccgcagtgtttaagcacttggctaacagtgtctcgaaacacggcctgtcaaatgtgtcgcgttatataccgcacgcgcacgcagtggcgtagtcgccttaacctgtggccggagatggagcgccaagaaatttttgaactgtttttgctgatgtctgtggtggtggctgggcttgtcggcgtggcgttgtgcacctggacgctcctggtcatcctaactgctcctgcgggaacattctcccccggggccgtgctcggttttctctgcttctttgggttttaccaaatatttattgtgtttgcatttggcggcatatgccgcgtaagtggcactgtgagggcattatacgcggcaaataacacccgggtgaccgtactgccataccgacggccgcgccggccaaccgcgaacgaagataacatcgaattgacggtccttgtcggacccgcgggcgggacggacgaggagcccacggacgagtcatctgaaggagacgtcgcctctggagacaaagaacgtgacggttcatccggagacgagccggacgggggcccgaacgaccgtgcgggacttagggggacagcgcgtaccgacctatgcgcgcccacaaaaaagccggtgcggaaaaatcatccaaaaaacaacggttga

SEQ ID NO: 19 - protein, 244 aa, Artificial Sequence
  UCHT1_VH-linker- UCHT1_VL
DIQMTQSPSSLSASVGNRVTITCRASQDIRNYLNWYQQKPGKAPKLLIYYTSRLESGVPSRFSGSGSGTDYTLTISSLQPEDFATYYCQQGNTLPWTFGQGTKVEIKSGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVS

SEQ ID NO: 20 - DNA, 795 bp, Artificial Sequence
  UCHT1_VH-linker- UCHT1_VL
atggaaaccgacaccctgctgctgtgggtgctgctcgtgtggattcctggcagcaccggcgacatccagatgacccagagccctagcagcctgagcgccagcgtgggcaacagagtgaccatcacctgtcgggccagccaggacatccggaactacctgaactggtatcagcagaagcccggcaaggcccccaagctgctgatctactacaccagcagactggaaagcggcgtgcccagcagattttccggcagcggctctggcaccgactacaccctgacaatcagcagcctgcagcccgaggacttcgccacctactattgccagcagggcaacaccctgccctggacctttggccagggcaccaaggtggaaatcaagagcggcggaggcggatctggcggagggggaagtggcgggggaggatctgaagtgcagctggtggaatctggcggcggactggtgcagcctggcggatctctgagactgagctgtgccgccagcggctacagcttcaccggctacaccatgaactgggtgcgccaggcccctggcaagggactggaatgggtggccctgatcaacccctacaagggcgtgtccacctacaaccagaagttcaaggatcggttcaccatcagcgtggacaagagcaagaacaccgcctacctgcagatgaacagcctgagagccgaggacaccgccgtgtactactgtgccagaagcggctactacggcgacagcgactggtacttcgacgtgtggggccagggaaccctcgtgaccgtgtctagc

SEQ ID NO: 21 - protein, 244 aa, Artificial Sequence
  anti-TCR YTH
DFMLTQPHSVSESPGKTVIISCTLSSGNIENNYVHWYQQRPGRAPTTVIFDDDKRPDGVPDRFSGSIDRSSNSASLTISGLQTEDEADYYCHSYVSSFNVFGGGTKLTVLSGGGGSGGGGSGGGGSEVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVS

SEQ ID NO: 22 - DNA, 792 bp, Artificial Sequence
  anti-TCR YTH
atggagaccgacaccctgctgctgtgggtgctgctgctgtgggtgccaggcagcaccggcgacttcatgctgacccagccccacagcgtgagcgagagcccaggcaagaccgtgatcatcagctgcaccctgagcagcggcaacatcgagaacaactacgtgcactggtaccagcagcggccaggccgggccccaaccaccgtgatcttcgacgacgacaagcggcccgacggcgtgcccgaccgcttcagcggcagcatcgaccggagcagcaacagcgccagcctgaccatcagcggcctgcagaccgaggacgaggccgactactactgccacagctacgtgagcagcttcaacgtgttcggcggcggcaccaagctgaccgtgctgagcggtggcggtggcagcggcggcggcggaagcggaggtggtggcagcgaggtgcagctgctggagagcggcggcggcctggtgcagcccggcggcagcctgcggctgagctgcgccgccagcggcttcaccttcagcagcttccccatggcctgggtgcggcaggccccaggcaagggcctggagtgggtgagcaccatcagcaccagcggcggccggacctactaccgggacagcgtgaagggccggttcaccatcagccgggacaacagcaagaacaccctgtacctgcagatgaacagcctgcgggccgaggacaccgccgtgtactactgcgccaagttccggcagtacagcggcggcttcgactactggggacagggcaccctggtgaccgtgagc

SEQ ID NO: 23 - protein, 241 aa, Artificial Sequence
  TR-66 anti-TCR
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKR

SEQ ID NO: 24 - DNA, 786 bp, Artificial Sequence
  TR-66 anti-TCR
atggagaccgacaccctgctgctgtgggtgctgctgctgtgggtgcccggcagcaccggcgacatcaagctgcagcagagcggcgccgagctggcccggcctggagccagcgtgaagatgagctgcaagaccagcggctacaccttcacccggtacaccatgcactgggtgaagcagcggcctggccagggcctggagtggatcggctacatcaaccccagccgcggctacaccaactacaaccagaagttcaaggacaaggccaccctgaccaccgacaagagcagcagcaccgcctacatgcagctgagcagcctgaccagcgaggacagcgccgtgtactactgcgcccggtactacgacgaccactactgcctggactactggggccagggcaccaccctgaccgtgagcagcggtggcggtggcagcggcggcggcggaagcggaggtggtggcagcgacatccagctgacccagagccccgccatcatgagcgccagccctggcgagaaggtgaccatgacctgccgggccagcagcagcgtgagctacatgaactggtaccagcagaagagcggcaccagccctaagcggtggatctacgacaccagcaaggtggccagcggcgtgccctatcggttcagcggcagcggcagcggcaccagctacagcctgaccatcagcagcatggaggccgaggacgccgccacctactactgccagcagtggagcagcaatcctctgaccttcggcgccggcaccaagctggagctgaagcggtcg

SEQ ID NO: 25 - protein, 260 aa, Artificial Sequence
  MHC class II complex binding domain
MGWSCIILFLVATATGVHSQIVLTQSPAIMSASPGEKVTMTCSASSIVRYMYWFQQKPGSSPRLLIYDTSSLSSGVPVRFSGSGSGTSYSLTISRMEAEDAATYFCQQWSSYPLTFGGGTKLEIKRSGGGGSGGGGSGGGGSEVKLEESGGGLVQPGGSMKLSCVASGFTFSNSWMNWVRQSPEKGLEWVAEIRLKSNNYATRYAESVKGRFTISRDDSKSSVYLQMNNLRSEDTAIYYCTPLSYSFDYWGQTTVTVSTA

SEQ ID NO: 26 - DNA, 780 bp, Artificial Sequence
  MHC class II complex binding domain
atgggctggagctgtatcatcctttttctagtagcaactgcaaccggtgtacactctcaaattgttcttactcagtctcccgccataatgagcgcgtcacccggagagaaagtgacaatgacttgctcagcctcatccatagtacgatacatgtattggttccagcagaaaccaggctcatcaccgagactgttgatttacgatacttccagtctgtctagtggtgtgccggtcagattctcagggtccggctctggtacctcttattctctcaccatatcacgcatggaggctgaagacgctgccacatatttctgccagcaatggagttcttaccctcttacgttcgggggcgggactaagttggaaatcaaaagatcaggagggggcggtagcggaggtggcggctcaggagggggtggatcagaggtaaaactggaggaaagcggtggtggactggtacaaccagggggctccatgaagctttcttgtgttgcgagtggattcacgttctcaaattcctggatgaactgggtgcgacaaagccccgaaaaggggcttgaatgggtcgctgaaatccgattgaaatccaataactacgccactcggtacgcagagtctgtaaagggtcgcttcacgattagcagggatgattccaaaagttccgtttatttgcaaatgaataatcttcggtctgaggacacggcgatttattactgcacccccctcagttattcctttgattattggggtcaaacaacagtgacggtgtcaacggcc

SEQ ID NO: 27 - protein, 6 aa, Artificial Sequence
  Linker
GGSGGS

SEQ ID NO: 28 - protein, 7 aa, Artificial Sequence
  Linker
SGSGSGS

SEQ ID NO: 29 - protein, 10 aa, Artificial Sequence
  Linker
GGGGSGGGGS

SEQ ID NO: 30 - protein, 10 aa, Artificial Sequence
  Linker
GSGSGSGSGS

SEQ ID NO: 31 - protein, 12 aa, Artificial Sequence
  Linker
GGSGGSGGSGGS

SEQ ID NO: 32 - protein, 15 aa, Artificial Sequence
  Linker
GGGGSGGGGSGGGGS

SEQ ID NO: 33 - protein, 20 aa, Artificial Sequence
  Signal peptide
METDTLLLWVLLVWIPGSTG

SEQ ID NO: 34 - protein, 445 aa, Artificial Sequence
  UCHT1-US11
DIQMTQSPSSLSASVGNRVTITCRASQDIRNYLNWYQQKPGKAPKLLIYYTSRLESGVPSRFSGSGSGTDYTLTISSLQPEDFATYYCQQGNTLPWTFGQGTKVEIKSGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSDPMPELSLTLFDEPPPLVETEPLPPLPDVSEYRVEYSEARCVLRSGGRLEALWTLRGNLSVPTPTPRVYYQTLEGYADRVPTPVEDISESLVAKRYWLRDYRVPQRTKLVLFYFSPCHQCQTYYVECEPRCLVPWVPLWSSLEDIERLLFEDRRLMAYYALTIKSAQYTLMMVAVIQVFWGLYVKGWLHRHFPWMFSDQW

SEQ ID NO: 35 - DNA, 1335 bp, Artificial Sequence
  UCHT1-US11
gacatccagatgacccagagccctagcagcctgagcgccagcgtgggcaacagagtgaccatcacctgtcgggccagccaggacatccggaactacctgaactggtatcagcagaagcccggcaaggcccccaagctgctgatctactacaccagcagactggaaagcggcgtgcccagcagattttccggcagcggctctggcaccgactacaccctgacaatcagcagcctgcagcccgaggacttcgccacctactattgccagcagggcaacaccctgccctggacctttggccagggcaccaaggtggaaatcaagagcggcggaggcggatctggcggagggggaagtggcgggggaggatctgaagtgcagctggtggaatctggcggcggactggtgcagcctggcggatctctgagactgagctgtgccgccagcggctacagcttcaccggctacaccatgaactgggtgcgccaggcccctggcaagggactggaatgggtggccctgatcaacccctacaagggcgtgtccacctacaaccagaagttcaaggatcggttcaccatcagcgtggacaagagcaagaacaccgcctacctgcagatgaacagcctgagagccgaggacaccgccgtgtactactgtgccagaagcggctactacggcgacagcgactggtacttcgacgtgtggggccagggaaccctcgtgaccgtgtctagcgatcctatgcccgagctgagcctgaccctgttcgacgagccaccacccctggtggagaccgagcccctgccacccctgcccgacgtgagcgagtaccgggtggagtacagcgaggcccggtgcgtgctgcggagcggcggccggctggaggccctgtggaccctgcggggcaacctgagcgtgcccaccccaaccccacgggtgtactaccagaccctggagggctacgccgaccgggtgcccaccccagtggaggacatcagcgagagcctggtggccaagcggtactggctgcgggactaccgggtgccccagcggaccaagctggtgctgttctacttcagcccctgccaccagtgccagacctactacgtggagtgcgagccccggtgcctggtgccctgggtgcccctgtggagcagcctggaggacatcgagcggctgctgttcgaggaccggcggctgatggcctactacgccctgaccatcaagagcgcccagtacaccctgatgatggtggccgtgatccaggtgttctggggcctgtacgtgaagggctggctgcaccggcacttcccctggatgttcagcgaccagtgg

SEQ ID NO: 36 - protein, 112 aa, Artificial Sequence
  CD3z endodomain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 368 aa, Artificial Sequence
  4-1BB and CD3z endodomains
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 38 - protein, 152 aa, Artificial Sequence
  CD28 and CD3z endodomains
SKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 188 aa, Artificial Sequence
  CD28, OX40 and CD3z endodomains
SKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 389 aa, Artificial Sequence
  UCHT1-E19
DIQMTQSPSSLSASVGNRVTITCRASQDIRNYLNWYQQKPGKAPKLLIYYTSRLESGVPSRFSGSGSGTDYTLTISSLQPEDFATYYCQQGNTLPWTFGQGTKVEIKSGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSDPTQKVDFKEPACNVTFAAEANECTTLIKCTTEHEKLLIRHKNKIGKYAVYAIWQPGDTTEYNVTVFQGKSHKTFMYTFPFYEMCDITMYMSKQYKLWPPQNCVENTGTFCCTAMLITVLALVCTLLYIKYKSRRSFIEEKKMP

SEQ ID NO: 41 - protein, 324 aa, Artificial Sequence
  antiCD3 VHH-US11
EVQAESGGGSVQTGGSLRLSCAYTASSVCMAWFRQAPGKEREGVAVTREGLTKTGYADSVKGRFAISQDYAKKTLYLQMSSLKPEDTARYYCAARPTSPCTVDGELLASTYNYWGQGTQVTVSSDPMPELSLTLFDEPPPLVETEPLPPLPDVSEYRVEYSEARCVLRSGGRLEALWTLRGNLSVPTPTPRVYYQTLEGYADRVPTPVEDISESLVAKRYWLRDYRVPQRTKLVLFYFSPCHQCQTYYVECEPRCLVPWVPLWSSLEDIERLLFEDRRLMAYYALTIKSAQYTLMMVAVIQVFWGLYVKGWLHRHFPWMFSDQW

SEQ ID NO: 42 - protein, 126 aa, Artificial Sequence
  anti-CD3 VHH
EVQAESGGGSVQTGGSLRLSCAYTASSVCMAWFRQAPGKEREGVAVTREGLTKTGYADSVKGRFAISQDYAKKTLYLQMSSLKPEDTARYYCAARPTSPCTVDGELLASTYNYWGQGTQVTVSSDP

SEQ ID NO: 43 - protein, 142 aa, Artificial Sequence
  adenovirus E19
TQKVDFKEPACNVTFAAEANECTTLIKCTTEHEKLLIRHKNKIGKYAVYAIWQPGDTTEYNVTVFQGKSHKTFMYTFPFYEMCDITMYMSKQYKLWPPQNCVENTGTFCCTAMLITVLALVCTLLYIKYKSRRSFIEEKKMP

SEQ ID NO: 44 - DNA, 426 bp, Artificial Sequence
  adenovirus E19
acccagaaggtggacttcaaagaacccgcctgcaacgtgacctttgccgccgaggccaatgagtgcaccacactgatcaagtgtaccaccgagcacgagaagctgctgatccggcacaagaacaagatcgggaagtacgccgtgtacgccatctggcagcctggcgataccacagagtacaatgtgaccgtgttccagggcaagagccacaagaccttcatgtacacctttccgttctacgagatgtgcgacatcacgatgtacatgagcaagcagtacaagctgtggcctcctcagaactgcgtcgagaataccggcaccttctgctgcaccgccatgctgattacagtgctggccctcgtgtgcaccctgctgtacatcaagtacaagagccggcggagcttcatcgaagagaagaagatgccc

SEQ ID NO: 45 - protein, 267 aa, Artificial Sequence
  UCHT1-E19
EVQAESGGGSVQTGGSLRLSCAYTASSVCMAWFRQAPGKEREGVAVTREGLTKTGYADSVKGRFAISQDYAKKTLYLQMSSLKPEDTARYYCAARPTSPCTVDGELLASTYNYWGQGTQVTVSDPTQKVDFKEPACNVTFAAEANECTTLIKCTTEHEKLLIRHKNKIGKYAVYAIWQPGDTTEYNVTVFQGKSHKTFMYTFPFYEMCDITMYMSKQYKLWPPQNCVENTGTFCCTAMLITVLALVCTLLYIKYKSRRSFIEEKKMP